(19) United States
(12) Patent Application Publication
(10) Pub. No.: US 2017/0348288 A1
(43) Pub. Date: Dec. 7, 2017

(54) DOSING REGIMENS FOR BETA-3 ADRENOCEPTOR AGONISTS AND ANTI-MUSCARINIC AGENTS FOR THE TREATMENT AND PREVENTION OF LOWER URINARY TRACT SYMPTOMS AND OVERACTIVE BLADDER

(71) Applicant: VELICEPT THERAPEUTICS, INC., Malvern, PA (US)

(72) Inventors: Eliot OHLSTEIN, Glennmoore, PA (US); Raymond E. STEVENS, JR., West Chester, PA (US); H. Jeffrey WILKINS, Sellersville, PA (US)

(21) Appl. No.: 15/614,531

(22) Filed: Jun. 5, 2017

Related U.S. Application Data
(60) Provisional application No. 62/345,283, filed on Jun. 3, 2016.
(60) Provisional application No. 62/345,655, filed on Jun. 3, 2016.

(51) Int. Cl. A61K 31/426; A61K 31/445; A61K 31/439; A61K 31/4025; A61K 31/222; A61K 31/216; A61K 31/137; A61K 31/46; A61J 1/03

(52) U.S. Cl. CPC A61K 31/426; A61K 31/46; A61K 31/445; A61K 31/439; A61J 1/03; A61K 31/222; A61K 31/216; A61K 31/137; A61K 31/4025

(57) ABSTRACT

Embodiments relate to pharmaceutical compositions, comprising one or more beta-3 adrenoceptor agonists that are useful for the treatment of lower urinary tract symptoms such as, for example, overactive bladder, prostate disorders and methods for treating lower urinary tract symptoms utilizing the pharmaceutical compositions, comprising one or more beta-3 adrenoceptor agonists. In some embodiments, the pharmaceutical compositions, comprising one or more beta-3 adrenoceptor agonists comprise pulsatile drug delivery systems. Embodiments further relate to a dosing regimen, comprising beta-3 adrenoceptor agonists and anti-muscarinic agents that are both useful for the treatment of lower urinary tract symptoms; methods for treating lower urinary tract symptoms methods utilizing the dosing regimens, comprising beta-3 adrenoceptor agonists anti-muscarinic agents; and consumer packaging, comprising both beta-3 adrenoceptor agonists and anti-muscarinic agents packaged and arranged to ensure patient compliance.

The present application claims the benefit of U.S. Provisional Application No. 62/345,283, filed Jun. 3, 2016; and U.S. Provisional Application No. 62/345,655, filed Jun. 3, 2016; the disclosures of which are hereby incorporated by reference in their entirety.

Embodiments of the present invention relate to pharmaceutical compositions comprising beta-3 adrenoceptor agonists that are useful for the treatment of lower urinary tract symptoms such as, for example, overactive bladder and prostate disorders and methods for treating lower urinary tract symptoms utilizing the pharmaceutical compositions, comprising beta-3 adrenoceptor agonists. Certain embodiments also relate to pharmaceutical compositions comprising one or more beta-3 adrenoceptor agonists comprising pulsatile drug delivery systems. Certain embodiments also relate to dosing regimens comprising beta-3 adrenoceptor agonists and anti-muscarinic agents that are both useful for the treatment of lower urinary tract symptoms such as, for example, overactive bladder and prostate disorders; methods for treating lower urinary tract symptoms and methods utilizing the dosing regimens comprising beta-3 adrenoceptor agonists and anti-muscarinic agents; and consumer packaging comprising both beta-3 adrenoceptor agonists and anti-muscarinic agents packaged and arranged to ensure patient compliance.

Agonist-induced desensitization of G-protein coupled receptors (GPCRs) is well documented. In particular, agonist-induced desensitization of the beta-1 and beta-2 adrenoceptors is well studied. For many disease processes, GPCR desensitization is thought to contribute to the disease process or limit the effect of therapeutic agents. Prolonged and constant exposure of the receptor system molecule to a drug may result in receptor down-regulation. Down-regulation occurs when there is a decrease in the number of receptor system molecules on the cell, thus decreasing the response to continued administration of the therapeutic agent. In addition, more drug may often be needed over time to achieve the same therapeutic response.

A new treatment for overactive bladder and lower urinary tract symptoms (hereinafter “LUTS”) are the beta-3 adrenoceptors agonists such as, for example, amibegron (SR-58,611A, ethyl 2-(((S)-7-(R)-2-(3-chlorophenyl)-2-hydroxyethyl)amino)-5,6,7,8-tetrahydronaphthalen-2-yl) oxy)acetate); mirabegron (YM-178, (R)-2-(2-aminothiazol-4-yl)-N-(4-(2-((2-hydroxy-2-phenylethyl) amino)ethyl)phenyl)acetamide); ritobegron (KUC-7483, (-)-ethyl 2-[4-(2-{[(1S,2R)-2-hydroxy-2-(4-hydroxyphenyl)-1-methyl ethyl]amino}ethyl)-2,5-dimethylphenyloxy]acetate monohydrochloride); vibegron (MK-4618, (6S)-N-[4-[[(2S,5R)-5-[(R)-hydroxy (phenyl)methyl]pyrrolidin-2-yl]methyl]phenyl]-4-oxo-7,8-dihydro-6H-pyrrolo[1,2-a]pyrimidine-6-carboxamide); solabegron (3-[3-[2-[ [(2R)-2-(3 -chlorophenyl)-2-hydroxyethyl]amino]ethylamino]phenyl]benzoic acid); L-742,791 ((S)-N-[4-[2-[[3-(4-hydroxyphenoxy)-2-hydroxypropyl]amino]ethyl]phenyl]-4-iodobenzenesulfonamide); L-796,568 (benzenesulfonamide, N-(4-(2-(((2R)-2-hydroxy-2-(3-pyridinyl)ethyl)amino)ethyl)phenyl)-4-(4-(4-(trifluoromethyl) phenyl)-2-thiazolyl)-, dihydrochloride); TRK-380; LY-368,842 ((S)-2-[4- [2-[3-(indol-2-[14C]-4-yloxy)-2-hydroxypropyl amino]-2-methylpropyl]-phenoxy]pyridine-5-carboxamide) and Ro40-2148. United States Patent Publication No. 20130172277A1 describes and claims the use of mirabegron as a combination therapy with an anti-muscarinic agent for the treatment of overactive bladder.

The question arises whether chronic long-term use of a beta-3 adrenoceptor agonist may be limited by beta-3 receptor desensitization. It is conceivable that like the beta-2 adrenoceptor in airway smooth muscle, continuous, prolonged administration of a beta-3 adrenoceptor agonist will elicit beta-3 receptor desensitization in bladder smooth muscle. Prolonged exposure of a beta-3 adrenoceptor agonist may result in a decrease in the number of beta-3 receptors, a decrease binding affinity or diminish post-receptor signal transduction mechanisms and second messenger signaling, resulting in a diminished therapeutic response.

There are examples in the literature where beta-3 adrenoceptors become desensitized following exposure to beta-3 agonists in some cell types. For example, down-regulation of murine adipocyte beta-3 adrenoceptor was reported in response the selective beta-3 agonist CL316,243. in addition, that study also demonstrated that the functional response was attenuated following prolonged exposure to the beta-3 agonist. Beta-3 receptor desensitization has also been reported in human cells. Human beta-3 adrenoceptors transfected into human embryonic kidney cells were sensitive to beta-3 agonist-induced desensitization.

A further new treatment for LUIS is a class of compounds known as anti-muscarinic agents. Examples of suitable antimuscarinic agents for use in the present application include tolterodine ((S)-2-(3-(diisopropylamino)-1-phenylpropyl)-4-methylphenol), oxybutynin (4-(diethylamino)but-2-yn-1-yl 2-cyclohexyl-2-hydroxy-2-phenyl acetate), trospium (spiro[8-azoniabicyclo [3.2.1] octane-8, 1′-azolidin-1-ium] -3 -yl 2-hydroxy-2,2-diphenyl acetate; chloride), solifenacin ([(3R)-1-azabicyclo[2.2.2]octan-3-yl] (1S)-1-phenyl-3,4-dihydro-1H-isoquinoline-2-carboxylate), darifenacin (2-[(3S)-1-[2-(2,3 -dihydro-1-benzofuran-5-yl)ethyl]pyrrolidin-3 -yl]-2,2-diphenylacetamide), propiverine ((1-methylpiperidin-4-yl) 2,2-diphenyl-2-propoxyacetate), fesoterodine ([2-[(1R)-3-[di(propan-2-yl)amino]-1-phenylpropyl]-4-(hydroxymethyl)phenyl] 2-methylpropanoate), and pharmaceutically acceptable salts thereof

Therefore, it is desired to provide pharmaceutical compositions capable of delivering beta-3 adrenoceptor agonists that both provide a patient in need of treatment with a desired blood plasma Cₘₐₓ and AUC while also minimizing desensitization of the beta-3 adrenoceptors, thus maximizing therapeutic benefit.

### SUMMARY

In one embodiment the present application describes a pharmaceutical composition comprising a therapeutically effective amount of one or more beta-3 adrenoceptor agonists, wherein the pharmaceutical composition releases at least two pulses of one or more beta-3 adrenoceptor agonists, wherein a first pulse of one or more beta-3 adrenoceptor agonists achieves a first target Cₘₐₓ, a second pulse of one or more beta-3 adrenoceptor agonists achieves a second target Cₘₐₓ, a first target Cₘᵢₙ is achieved between the first pulse and the second pulse and a second Cₘᵢₙ is achieved after the second pulse. Other embodiments of the present application describe pharmaceutical compositions, further comprising one or more additional therapeutic agents useful for the treatment of LUTS, wherein the one or more additional therapeutic agents are antimuscarinic agents, alpha adrenoceptor blockers, 5-alpha reductases or phosphodiesterase-5 inhibitors and the one or more additional therapeutic agents may be administered prior to, simultaneously with, or following the administration of the pharmaceutical composition comprising one or more beta-3 adrenoceptor agonists. In embodiments, the beta-3 adrenoceptor agonist is selected from amibegron; mirabegron; ritobegron; vibegron; L-742,791; L-796,568; TRK-380, LY-368,842; Ro40-2148 and pharmaceutically acceptable salts thereof. In embodiments, the beta-3 adrenoceptor agonist is selected from amibegron; mirabegron; ritobegron; and vibegron. In embodiments, the first target Cₘₐₓ and the second target Cₘₐₓ are different. In embodiments, the first pulse and the second pulse are different amounts of the one or more beta-3 adrenoceptor agonists.

In one embodiment, the present application describes a pharmaceutical composition for the delivery of one or more beta-3 adrenoceptor agonists, comprising at least one immediate release composition, comprising one or more beta-3 adrenoceptor agonists and at least one pharmaceutically acceptable carrier or diluent; and at least one modified release composition, comprising one or more beta-3 adrenoceptor agonists and at least one pharmaceutically acceptable carrier or diluent. Other embodiments of the present application describe pharmaceutical compositions, further comprising one or more additional therapeutic agents useful for the treatment of LUTS, wherein the one or more additional therapeutic agents are antimuscarinic agents, alpha adrenoceptor blockers, 5-alpha reductases or phosphodiesterase-5 inhibitors and the one or more additional therapeutic agents may be administered prior to, simultaneously with, or following the administration of the pharmaceutical composition comprising one or more beta-3 adrenoceptor agonists. In embodiments, the beta-3 adrenoceptor agonist is selected from amibegron; mirabegron; ritobegron; vibegron; L-742,791; L-796,568; TRK-380, LY-368,842; Ro40-2148 and pharmaceutically acceptable salts thereof. In embodiments, the beta-3 adrenoceptor agonist is selected from amibegron; mirabegron; ritobegron; and vibegron.

In one embodiment the present application describes a method for treating LUTS, comprising: administering a pharmaceutical composition for the delivery of one or more beta-3 adrenoceptor agonists, comprising: an immediate release composition, comprising one or more beta-3 adrenoceptor agonists and at least one pharmaceutically acceptable carrier or diluent; and a modified release composition, comprising one or more beta-3 adrenoceptor agonists and at least one pharmaceutically acceptable carrier or diluent to a patient in need thereof. Additional embodiments describe methods, wherein the pharmaceutical composition is administered every other day (QOD); once a day (QD), twice a day (BID) or three times a day (TID) to a patient in need thereof. Further embodiments describe methods, further comprising administering one or more additional therapeutic agents useful for the treatment of LUTS, wherein the one or more additional therapeutic agents are selected from the groups consisting of: antimuscarinic agents; alpha adrenoceptor blockers; 5-alpha reductases; and phosphodiesterase-5 inhibitors, wherein the one or more additional therapeutic agents may be administered prior to, simultaneously with, or following the administration of the pharmaceutical composition comprising one or more beta-3 adrenoceptor agonists. In embodiments, the beta-3 adrenoceptor agonist is selected from amibegron; mirabegron; ritobegron; vibegron; L-742,791; L-796,568; TRK-380, LY-368,842; and Ro40-2148 and pharmaceutically acceptable salts thereof. In embodiments, the beta-3 adrenoceptor agonist is selected from amibegron; mirabegron; ritobegron; and vibegron.

In one embodiment the present application describes a method for treating LUTS, comprising: administering a pharmaceutical composition, comprising a therapeutically effective amount of one or more beta-3 adrenoceptor agonists, wherein the pharmaceutical composition releases at least two pulses of one or more beta-3 adrenoceptor agonists, wherein a first pulse of one or more beta-3 adrenoceptor agonists achieves a first target Cₘₐₓ, a second pulse of one or more beta-3 adrenoceptor agonists achieves a second target Cₘₐₓ, a first target Cₘᵢₙ is achieved between the first pulse and the second pulse and a second Cₘᵢₙ is achieved after the second pulse. Further embodiments describe methods that further comprise administering one or more additional therapeutic agents useful for the treatment of LUTS, wherein the one or more additional therapeutic agents are selected from the groups consisting of: antimuscarinic agents; alpha adrenoceptor blockers; 5-alpha reductases; and phosphodiesterase-5 inhibitors, wherein the one or more additional therapeutic agents may be administered prior to, simultaneously with, or following the administration of the pharmaceutical composition comprising one or more beta-3 adrenoceptor agonists. Still further embodiments describe methods, wherein the pharmaceutical composition is administered every other day (QOD); once a day (QD), twice a day (BID) or three times a day (TID) to a patient in need thereof In embodiments, the beta-3 adrenoceptor agonist is selected from amibegron; mirabegron; ritobegron; vibegron; L-742,791; L-796,568; TRK-380, LY-368,842; and Ro40-2148 and pharmaceutically acceptable salts thereof. In embodiments, the beta-3 adrenoceptor agonist is selected from amibegron; mirabegron; ritobegron; and vibegron.

In one embodiment the present application describes a once-daily treatment for LUTS that achieves a desired blood serum Cₘₐₓ while also not desensitizing the beta-3 adrenoceptor, comprising: a pharmaceutical composition, comprising a therapeutically effective amount of one or more beta-3 adrenoceptor agonists, wherein the pharmaceutical composition releases at least two pulses of one or more beta-3 adrenoceptor agonists, wherein a first pulse of one or more beta-3 adrenoceptor agonists achieves a first target Cₘₐₓ, a second pulse of one or more beta-3 adrenoceptor agonists achieves a second target Cₘₐₓ, a first target Cₘᵢₙ is achieved between the first pulse and the second pulse and a second Cₘᵢₙ is achieved after the second pulse. Further embodiments describe treatments further comprising administering one or more additional therapeutic agents useful for the treatment of LUTS, wherein the one or more additional therapeutic agents are selected from the groups consisting of: antimuscarinic agents; alpha adrenoceptor blockers; 5-alpha reductases; and phosphodiesterase-5 inhibitors, wherein the one or more additional therapeutic agents may be administered prior to, simultaneously with, or following the administration of the pharmaceutical composition comprising one or more beta-3 adrenoceptor agonists. In embodiments, the beta-3 adrenoceptor agonist is selected from amibegron; mirabegron; ritobegron; vibegron; L-742,791; L-796,568; TRK-380, LY-368,842; Ro40-2148 and pharmaceutically acceptable salts thereof In embodiments, the beta-3 adrenoceptor agonist is selected from amibegron; mirabegron; ritobegron; and vibegron.

In one embodiment the present application describes a once-daily treatment for LUTS that achieves a desired blood serum Cₘₐₓ while also not desensitizing the beta-3 adrenoceptor, comprising: at least one immediate release composition, comprising one or more beta-3 adrenoceptor agonists and at least one pharmaceutically acceptable carrier or diluent; and at least one modified release composition, comprising one or more beta-3 adrenoceptor agonists and at least one pharmaceutically acceptable carrier or diluent. Further embodiments describe treatments, further comprising administering one or more additional therapeutic agents useful for the treatment of LUTS, wherein the one or more additional therapeutic agents are selected from the groups consisting of: antimuscarinic agents; alpha adrenoceptor blockers; 5-alpha reductases; and phosphodiesterase-5 inhibitors, wherein the one or more additional therapeutic agents may be administered prior to, simultaneously with, or following the administration of the pharmaceutical composition comprising one or more beta-3 adrenoceptor agonists In embodiments, the beta-3 adrenoceptor agonist is selected from amibegron; mirabegron; ritobegron; vibegron; L-742,791; L-796,568; TRK-380, LY-368,842; Ro40-2148 and pharmaceutically acceptable salts thereof. In embodiments, the beta-3 adrenoceptor agonist is selected from amibegron; mirabegron; ritobegron; and vibegron.

In one embodiment the present application describes a method of treating one or more symptoms of LUTS, comprising: administering a pharmaceutical composition, comprising a therapeutically effective amount of one or more beta-3 adrenoceptor agonists and at least one pharmaceutically acceptable diluent or carrier, wherein the one or more symptoms of LUTS are selected from the group consisting of: increased urinary urgency of urination, increase in urinary micturition frequency, urinary incontinence, painful urination, excessive passage of urine at night, poor stream, overactive bladder, hesitancy, terminal dribbling, incomplete voiding and overflow incontinence. Further embodiments describe methods, wherein the pharmaceutical composition will be administered in the morning or the pharmaceutical composition will be administered with a meal. Additional embodiments describe methods, wherein the improvement in the one or more symptoms of over active bladder is increased bladder volume as measured by void volume. In embodiments, the beta-3 adrenoceptor agonist is selected from amibegron; mirabegron; ritobegron; vibegron; solabegron; L-742,791; L-796,568; TRK-380; LY-368,842; Ro40-2148 and pharmaceutically acceptable salts thereof..In embodiments, the beta-3 adrenoceptor agonist is selected from amibegron; mirabegron; ritobegron; vibegron; L-742,791; L-796,568; TRK-380, LY-368,842; and Ro40-2148. In embodiments, the beta-3 adrenoceptor agonist is selected from amibegron; mirabegron; ritobegron; and vibegron.

In embodiments, the pharmaceutical composition reduces desensitization of beta-3 adrenoceptor or otherwise increases the therapeutic effect of beta-3 adrenoceptor agonist, particularly when compared to an immediate release formulation of beta-3 adrenoceptor agonist that may be given, for examples, twice daily. Desensitization occurs when the beta-3 adrenoceptor is not otherwise responsive to an agonist (or antagonist), is less responsive to an agonist (or antagonist), or the target tissue (e.g., the bladder) is not otherwise responsive or is less responsive to an agonist (or antagonist). Other embodiments of the present application relate to pharmaceutical compositions comprising a therapeutically effective amount of beta-3 adrenoceptor agonist that achieves a first target Cₘₐₓ of beta-3 adrenoceptor agonist, a second target Cₘₐₓ of beta-3 adrenoceptor agonist, a first target Cₘᵢₙ of beta-3 adrenoceptor agonist between the first target Cₘₐₓ, and the second target Cₘₐₓ and a second Cₘᵢₙ of beta-3 adrenoceptor agonist after the second target Cₘₐₓ. In embodiments, the first target Cₘₐₓ is less than the equivalent Cₘₐₓ of a 25 mg to 50 mg, a 25 mg to a 300 mg or 3 mg to 100 mg administration of a once daily, immediate release oral composition of beta-3 adrenoceptor agonist. In embodiments, the first target Cₘᵢₙ is less than the equivalent Cₘᵢₙ of a 25 mg to 50 mg, a 25 mg to 300 mg or a 3 mg to 100 mg administration of a once daily, immediate release oral composition of beta-3 adrenoceptor agonist. In embodiments, the second target Cₘₐₓ is less than the equivalent second target Cₘₐₓ of a 25 mg to 50 mg, a 25 mg to 300 mg or a 3 mg to 100 mg administration of a once daily immediate release oral composition of beta-3 adrenoceptor agonist. In embodiments, the second target Cₘᵢₙ is less than the equivalent second target Cₘᵢₙ of a 25 mg to 50 mg, a 25 mg to 300 mg or a 3 mg to 100 mg administration of a once daily immediate release oral composition of beta-3 adrenoceptor agonist. In embodiments, the pharmaceutical compositions reduce desensitization of beta-3 adrenoceptor, particularly when compared to an immediate release formulation of beta-3 adrenoceptor agonist that may be given, for example, once or twice daily. In embodiments, the pharmaceutical compositions achieve a plasma concentration [C] of beta-3 adrenoceptor agonist of about 1 μg/ml or below for a period of time of about 6 hours to about 9 hours during a 24 hour period. In embodiments, the pharmaceutical compositions achieve an AUC equivalent to the administration of about 25 mg to 50 mg, a 25 mg to 300 mg or a 3 mg to 100 mg of beta-3 adrenoceptor agonist in a once daily immediate release oral composition. In embodiments, the pharmaceutical composition achieves a target area under the curve (herein after AUC) over a 24 hour period. In embodiments, the pharmaceutical compositions may be administered once a day, once every two days, once every three days, once every four days, once every five days, once every six days or once every seven days to a patient in need thereof In embodiments, the beta-3 adrenoceptor agonist is selected from amibegron; mirabegron; ritobegron; vibegron; solabegron; L-742,791; L-796,568; TRK-380; LY-368,842; Ro40-2148 and pharmaceutically acceptable salts thereof In embodiments, the beta-3 adrenoceptor agonist is selected from amibegron; mirabegron; ritobegron; vibegron; L-742,791; L-796,568; TRK-380; LY-368,842; and Ro40-2148. In embodiments, the beta-3 adrenoceptor agonist is selected from amibegron; mirabegron; ritobegron; and vibegron.

Further embodiments are directed to the use of such pharmaceutical compositions for the treatment of diseases, including, but not limited to, lower urinary tract symptoms, obesity, type 2 diabetes, heart failure, irritable bowel syndrome and similar gastrointestinal disorders, pre-term labor, depression and anxiety. In embodiments, LUTS may be overactive bladder and/or prostate disorders.

Agonist-induced desensitization of G-protein coupled receptors (GPCRs) of the beta-3 adrenoceptor is not well studied. For many disease processes, GPCR desensitization is thought to contribute to the disease process or limit the effect of therapeutic agents. Prolonged exposure of the receptor system molecule to a drug may result in receptor down-regulation. Down-regulation occurs when there is a decrease in the number of receptor system molecules on the cell, thus decreasing the response to continued administration of the therapeutic agent. In addition, more drug may often be needed over time to achieve the same therapeutic response. Pharmaceutical compositions that increase the therapeutically effective properties of beta-3 adrenoceptor agonist, while otherwise minimizing such desensitization are described herein.

In one embodiment, the present application describes a dosing regimen for treating LUTS, comprising a.) administering a pharmaceutical composition comprising, a therapeutically effective amount of a beta-3 adrenoceptor agonist and at least one pharmaceutically acceptable carrier or diluent on days 1, 3, 5, 7, 9, 11, 13, 15, 17, 19. 21, 23, 25 and 27 of a 28 day dosing cycle; and b.) administering a pharmaceutical composition comprising, a therapeutically effective amount of anti-muscarinic agent and at least one pharmaceutically acceptable carrier or diluent on days 2, 4, 6, 8, 10, 12, 14, 16, 18, 20, 22, 24, 26 and 28 of a 28 day dosing cycle. In embodiments, the beta-3 adrenoceptor agonist is selected from amibegron; mirabegron; ritobegron; vibegron; solabegron; L-742,791; L-796,568; TRK-380; LY-368,842; Ro40-2148 and pharmaceutically acceptable salts thereof. In embodiments, the pharmaceutical composition may be an immediate release composition for oral administration. In embodiments, the pharmaceutical composition may be a pharmaceutical composition as described in the embodiments presented herein, including compositions with at least two pulses or compositions comprising an immediate release and a modified release composition.

In one embodiment, the present application describes a method for treating LUTS utilizing any one of the dosing regimens described herein. In one embodiment, a method of treating LUTS comprises administering a beta-3 adrenoceptor agonist and an antimuscarinic agent on alternating days. In embodiments, the beta-3 adrenoceptor agonist is selected from amibegron; mirabegron; ritobegron; vibegron; solabegron; L-742,791; L-796,568; TRK-380; LY-368,842; Ro40-2148 and pharmaceutically acceptable salts thereof

In one embodiment, the present application describes a consumer pack for treating LUTS, comprising at least one of beta-3 adrenoceptor agonist pharmaceutical composition, and at least one of the anti-muscarinic agent pharmaceutical compositions described herein and a packet, comprising 28 separate containers, wherein the beta-3 adrenoceptor agonist is provided for on days 1, 3, 5, 7, 9, 11, 13, 15, 17, 19, 21, 23, 25, and 27 and anti-muscarinic agent is provided for on days 2, 4, 6, 8, 10, 12, 14, 16, 18, 20, 22, 24, 26, and 28. In embodiments, the beta-3 adrenoceptor agonist is selected from amibegron; mirabegron; ritobegron; vibegron; solabegron; L-742,791; L-796,568; TRK-380; LY-368,842; Ro40-2148 and pharmaceutically acceptable salts thereof.

In any of the foregoing embodiments, the immediate release composition and the modified release composition are separate and distinct pharmaceutical compositions. In any of the foregoing embodiments, the immediate release composition and the modified release composition are separate and distinct pharmaceutical compositions.

To prevent or reduce beta-3 adrenoceptor desensitization, it is described herein that the therapeutic administration of a beta-3 adrenoceptor agonist occurs in a manner such that drug occupancy at the receptor occurs at levels that do not elicit significant receptor desensitization and pharmaceutical compositions that achieve the same.

It is well established in the GPCR field that prolonged occupancy of a receptor by an agonist can result in receptor desensitization. A method to limit this is to have the agonist off the receptor, and allow the receptor to recover from agonist occupancy. When examining an entire population of receptors, the entire population of receptors does not need to be unoccupied; fractional occupancy of the entire receptor population can still result in prevention of receptor desensitization and preservation of function. In other words, anything lower than 100% receptor occupancy may still allow some percentage of receptor resensitization.

Certain pharmaceutical compositions and methods of administration as described herein will not produce significant receptor desensitization, while ensuring the method of administration will optimize for the beta-3 adrenoreceptor stimulation, thus enabling the target tissue to benefit fully from the administered therapeutic agent. The therapeutic agent, in the present application, is one or more beta-3 adrenoceptor agonists and may be administered in a succession of at least two releases. The releases have a selected amplitude and duration so that the beta-3 adrenoreceptor will not down-regulate and the binding affinity of the receptor system molecule will not be diminished.

### BRIEF DESCRIPTION OF THE FIGURES

FIG. 1: Graphical illustration of a dual-pulse pharmaceutical composition that achieves a first target Cₘₐₓ, provides a period at a first target Cₘᵢₙ, achieves a second target Cₘₐₓ and finally provides a period at a second target Cₘᵢₙ.

FIG. 2: Cumulative concentration response curves (CCRC) to mirabegron performed after a one hour incubation to the EC₉₀ concentration of mirabegron and a period of washout using PSS. Two-way ANOVA to compare the curves gives p<0.001, with a Bonferroni post hoc test to compare individual points with comparable points on the vehicle incubation curve (triangles). *=p<0.05, **=p<0.01, ***=p<0.001.

FIG. 3: Cumulative concentration response curves (CCRC) to mirabegron performed after a three hour incubation to the EC₉₀ concentration of mirabegron and a period of washout using PSS. Two-way ANOVA to compare the curves gives p<0.001, with a Bonferroni post hoc test to compare individual points with comparable points on the vehicle incubation curve (triangles). *=p<0.05, **=p<0.01, ***=p<0.001

FIG. 4: Cumulative concentration response curves (CCRC) to CL-316,243 performed after a three hour incubation to the EC₉₀ concentration of CL-316 and a period of washout using PSS. Two-way ANOVA to compare the curves gives p<0.001, with a Bonferroni post hoc test to compare individual points with comparable points on the vehicle incubation curve (triangles). *=p<0.05, **=p<0.01, ***=p<0.001

FIG. 5: Example of a 28-day packet containing a beta-3 adrenoceptor agonist and an antimuscarinic agent in alternating containers for use in the present application.

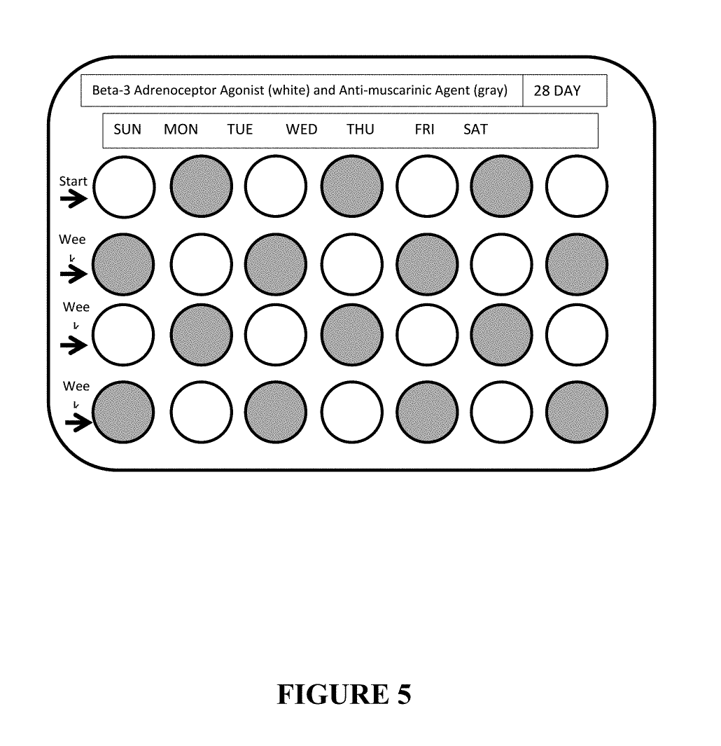

### DETAILED DESCRIPTION

### Definitions

As used herein, the term “about” means plus or minus 10% of a given value. For example, “about 50%” means in the range of 45%-55%.

As used herein the term “agonist” refers to a compound, the presence of which results in a biological activity of a receptor that is the same as the biological activity resulting from the presence of a naturally occurring ligand for the receptor.

The phrase “pharmaceutically acceptable” refers to molecular entities and compositions that are generally regarded as safe and nontoxic. In particular, pharmaceutically acceptable carriers, diluents or other excipients used in the pharmaceutical compositions of this application are physiologically tolerable, compatible with other ingredients, and do not typically produce an allergic or similar untoward reaction (for example, gastric upset, dizziness and the like) when administered to a patient. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The phrase “pharmaceutically acceptable salt(s),” as used herein, includes those salts of compounds of the application that are safe and effective for use in mammals and that possess the desired biological activity. Pharmaceutically acceptable salts include salts of acidic or basic groups present in compounds of the application or in compounds identified pursuant to the methods of the application. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the application can form pharmaceutically acceptable salts with various amino acids. Suitable base salts include, but are not limited to, aluminum, calcium, lithium, magnesium, potassium, sodium, zinc, iron and diethanolamine salts. Pharmaceutically acceptable base addition salts are also formed with amines, such as organic amines. Examples of suitable amines are N,N′-dibenzylethylenediamine, chloroprocaine, choline, diethanolamine, dicyclohexylamine, ethylenediamine, N-methylglucamine, and procaine.

As used herein the phrase “lower urinary tract symptoms” or “LUTS” refers to a group of medical symptoms, comprising increased urinary urgency of urination, increase in urinary micturition frequency, urinary incontinence, painful urination, excessive passage of urine at night, poor stream, overactive bladder, hesitancy, terminal dribbling, incomplete voiding and overflow incontinence.

As used herein the phrase “overactive bladder” or “OAB” refers to a group of medical symptoms, comprising urinary urgency, frequent urination, nocturia, urinating unintentionally and urge incontinence.

As used herein the phrase “beta-3 adrenoceptor agonists” refers to a class of molecules that are agonists against the beta-3 adrenergic receptor such as, for example amibegron; mirabegron; ritobegron; vibegron; solabegron; L-742,791; L-796,568; TRK-380; LY-368,842 and Ro40-2148.

As used herein the term “antimuscarinic agent” can be used interchangeably with the term “muscarinic receptor antagonist” (MRA) and is taken to mean a type of anticholinergic agent that blocks the activity of the muscarinic acetylcholine receptor such as, for example tolterodine, oxybutynin, trospium, solifenacin, darifenacin, propiverine, fesoterodine, and pharmaceutically acceptable salts thereof. Acetylcholine (often abbreviated ACh) is a neurotransmitter whose receptor is a protein found in synapses and other cell membranes. Besides responding to their primary neurochemical, neurotransmitter receptors can be sensitive to a variety of other molecules.

As used herein, the term “therapeutic” means an agent utilized to treat, combat, ameliorate, protect against or improve an unwanted condition or disease of a subject.

As used herein, the term “effective amount” refers to an amount that results in measurable inhibition of at least one symptom or parameter of a specific disorder or pathological process.

As used herein the term “therapeutically effective amount” of compositions of the application is a predetermined amount which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect or physician observes a change).

As used herein the terms “treat,” “treated,” or “treating” refer to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

As used herein the terms, “pulse,” “pulses,” “pulsed delivery,” or “pulsatile delivery device” refer to pharmaceutical compositions and methods of treatment wherein a therapeutic agent is delivered rapidly within a short period of time, as a result of a biological or external trigger, after a specific lag time.

As used herein the term “immediate release” refers to pharmaceutical compositions that release the active ingredient within a small period of time, typically less than 30 minutes.

As used herein the term “modified release” refers to pharmaceutical compositions that either release the active ingredient at a sustained and controlled release rate over a period of time such as, for example, 8 hours, 12 hours, 16 hours, and 24 hours or release the pharmaceutical dosage after a set time such as, for example, enteric-coated compositions that release the dosage in the intestinal track.

As used herein the phrase “drug delivery system” refers to any physical form, vehicle or composition that may be formulated to administer a therapeutic agent to a patient in need thereof such as, for example but not limited to the following: tablets, capsules, granules, powders, liquids, suspensions, suppositories, ointments, creams and aerosols.

As used herein, the term “desensitization” refers to a state wherein a receptor, specifically in the present application a beta-3 adrenoceptor, has been overexposed to an agonist for an extended period of time and an increased dosage of agonist must be administered to achieve a similar physiological response. It is a process whereby after prolonged agonist exposure, the receptor is uncoupled from its signaling ascade, and thus the biological effect of receptor activation is attenuated.

As used herein the terms “QD” and “q.d.” mean once a day (from the Latin quaque die).

As used herein the terms “QOD” and “q.o.d.” mean every other day (from the Latin quaque alters die).

As used herein the terms “BID” and “b.i.d.” mean twice a day (from the Latin bis in die).

As used herein the terms “TID” and t.i.d.” mean three times a day (from the Latin ter in die).

As used herein the terms “Cₘₐₓ” “Cₘᵢₙ,” “Tₘₐₓ,” and “Tₘᵢₙ” are terms used in pharmacokinetic analyses of the concentration of a drug against time. Cₘₐₓ is a term that refers to the maximum (or peak) plasma concentration that a drug achieves in a specified compartment or test area of the body after the drug has been administrated and prior to the administration of a second dose. Cₘₐₓ is the opposite of Cₘᵢₙ, which is the minimum (or trough) concentration that a drug achieves after dosing. Tₘₐₓ is the term used in pharmacokinetics to describe the time at which the Cₘₐₓ is observed and Tₘᵢₙ is the term used in pharmacokinetics to describe the time at which the Cₘₐₓ is observed after the drug has been administered and prior to the administration of a second dose.

As used herein the term “AUC” is the area under the curve (mathematically known as a definite integral) in a pharmacokinetic plot of the concentration of a drug against time.

### Embodiments

In one embodiment, the present application describes a pharmaceutical composition comprising: a therapeutically effective amount of one or more beta-3 adrenoceptor agonists, wherein the pharmaceutical composition releases at least two pulses of one or more beta-3 adrenoceptor agonists, wherein a first pulse of one or more beta-3 adrenoceptor agonists achieves a first target Cₘₐₓ, a second pulse of one or more beta-3 adrenoceptor agonists achieves a second target Cₘₐₓ, a first target Cₘᵢₙ is achieved between the first pulse and the second pulse and a second Cₘᵢₙ is achieved after the second pulse. In embodiments, the first target Cₘₐₓ and the second target Cₘₐₓ are different. In embodiments, the first pulse and the second pulse are different amounts of the one or more beta-3 adrenoceptor agonists. Other embodiments of the present application describe pharmaceutical compositions, further comprising one or more additional therapeutic agents useful for the treatment of LUTS, wherein the one or more additional therapeutic agents are antimuscarinic agents, alpha adrenoceptor blockers, 5-alpha reductases or phosphodiesterase-5 inhibitors and the one or more additional therapeutic agents may be administered prior to, simultaneously with, or following the administration of the pharmaceutical composition comprising one or more beta-3 adrenoceptor agonists. In embodiments, the beta-3 adrenoceptor agonist is selected from amibegron; mirabegron; ritobegron; vibegron; L-742,791; L-796,568; TRK-380, LY-368,842; Ro40-2148 and pharmaceutically acceptable salts thereof. In embodiments, the beta-3 adrenoceptor agonist is selected from amibegron; mirabegron; ritobegron; and vibegron. In certain embodiments, the antimuscarinic agent is selected from the group consisting of tolterodine, oxybutynin, trospium, solifenacin, darifenacin, propiverine, fesoterodine, and pharmaceutically acceptable salts thereof. In some embodiments, the target Cₘₐₓ achieves between about 50% and about 100% of the beta-3 adrenoceptor response as measured by urinary urgency, frequent urination, nocturia, urinating unintentionally, urge incontinence or bladder capacity. In some embodiments, the target Cₘᵢₙ achieves between about 50% and about 0% of the beta-3 adrenoceptor response as measured by urinary urgency, frequent urination, nocturia, urinating unintentionally, urge incontinence or bladder capacity. In some embodiments, the first pulse comprises about 12 mg to about 200 mg of one or more beta-3 adrenoceptor agonists. In some embodiments, the second pulse comprises about 12 mg to about 200 mg of one or more beta-3 adrenoceptor agonists. In embodiments, the pharmaceutical composition reduces desensitization of beta-3 adrenoceptor or otherwise increases the therapeutic effect of beta-3 adrenoceptor agonist, particularly when compared to an immediate release formulation of beta-3 adrenoceptor agonist that may be given, for examples, twice daily. Desensitization occurs when the beta-3 adrenoceptor is not otherwise responsive to an agonist, is less responsive to an agonist, or the target tissue (e.g., the bladder) is not otherwise responsive or is less responsive to an agonist. In embodiments, the first target Cₘₐₓ is less than the equivalent Cₘₐₓ of a 25 mg to 50 mg, a 25 mg to 300 mg or a 3 mg to 100 mg administration of a once daily, immediate release oral composition of beta-3 adrenoceptor agonist. In embodiments, the first target Cₘᵢₙ is less than the equivalent Cₘᵢₙ of a 25 mg to 50 mg, a 25 mg to 300 mg or a 3 mg to 100 mg administration of a once daily, immediate release oral composition of beta-3 adrenoceptor agonist. In embodiments, the second target Cₘₐₓ is less than the equivalent second target Cₘₐₓ of a 25 mg to 50 mg, a 25 mg to 300 mg or a 3 mg to 100 mg administration of a once daily immediate release oral composition of beta-3 adrenoceptor agonist. In embodiments, the second target Cₘᵢₙ is less than the equivalent second target Cₘᵢₙ of a 25 mg to 50 mg, a 25 mg to 300 mg or a 3 mg to 100 mg administration of a once daily immediate release oral composition of beta-3 adrenoceptor agonist. In embodiments, the pharmaceutical compositions reduce desensitization of beta-3 adrenoceptor, particularly when compared to an immediate release formulation of beta-3 adrenoceptor agonist that may be given, for example, once or twice daily. In embodiments, the pharmaceutical compositions achieve a plasma concentration [C] of beta-3 adrenoceptor agonist of about 1 μg/ml or below for a period of time of about 6 hours to about 9 hours during a 24 hour period. In embodiments, the pharmaceutical compositions achieve an AUC equivalent to the administration of about 25 mg to 50 mg, a 25 mg to 300 mg or a 3 mg to 100 mg of beta-3 adrenoceptor agonist in a once daily immediate release oral composition. In embodiments, the pharmaceutical composition achieves a target area under the curve (herein after AUC) over a 24 hour period.

In one embodiment, the present application describes a pharmaceutical composition for the delivery of one or more beta-3 adrenoceptor agonists, comprising: at least one immediate release composition, comprising one or more beta-3 adrenoceptor agonists and at least one pharmaceutically acceptable carrier or diluent; and at least one modified release composition, comprising one or more beta-3 adrenoceptor agonists and at least one pharmaceutically acceptable carrier or diluent. Other embodiments of the present application describe pharmaceutical compositions, further comprising one or more additional therapeutic agents useful for the treatment of LUTS, wherein the one or more additional therapeutic agents are antimuscarinic agents, alpha adrenoceptor blockers, 5-alpha reductases or phosphodiesterase- 5 inhibitors and the one or more additional therapeutic agents may be administered prior to, simultaneously with, or following the administration of the pharmaceutical composition comprising one or more beta-3 adrenoceptor agonists. n embodiments, the beta-3 adrenoceptor agonist is selected from amibegron; mirabegron; ritobegron; vibegron; L-742,791; L-796,568; TRK-380, LY-368,842; Ro40-2148 and pharmaceutically acceptable salts thereof. In embodiments, the beta-3 adrenoceptor agonist is selected from amibegron; mirabegron; ritobegron; and vibegron.In certain embodiments, the antimuscarinic agent is selected from the group consisting of tolterodine, oxybutynin, trospium, solifenacin, darifenacin, propiverine, fesoterodine, and pharmaceutically acceptable salts thereof In some embodiments, the at least one immediate release composition comprises about 12 mg to about 200 mg of one or more beta-3 adrenoceptor agonists. In some embodiments, the at least one modified release composition comprises about 12 mg to about 200 mg of one or more beta-3 adrenoceptor agonists. In embodiments, the pharmaceutical composition reduces desensitization of beta-3 adrenoceptor or otherwise increases the therapeutic effect of beta-3 adrenoceptor agonist, particularly when compared to an immediate release formulation of beta-3 adrenoceptor agonist that may be given, for examples, twice daily. Desensitization occurs when the beta-3 adrenoceptor is not otherwise responsive to an agonist, is less responsive to an agonist, or the target tissue (e.g., the bladder) is not otherwise responsive or is less responsive to an agonist. In embodiments, the first target Cₘₐₓ is less than the equivalent Cₘₐₓ of a 25 mg to 50 mg, a 25 mg to 300 mg or a 3 mg to 100 mg administration of a once daily, immediate release oral composition of beta-3 adrenoceptor agonist. In embodiments, the first target Cₘᵢₙ is less than the equivalent Cₘᵢₙ of a 25 mg to 50 mg, a 25 mg to 300 mg or a 3 mg to 100 mg administration of a once daily, immediate release oral composition of beta-3 adrenoceptor agonist. In embodiments, the second target Cₘₐₓ is less than the equivalent second target Cₘₐₓ of a 25 mg to 50 mg, a 25 mg to 300 mg or a 3 mg to 100 mg administration of a once daily immediate release oral composition of beta-3 adrenoceptor agonist. In embodiments, the second target Cₘᵢₙ is less than the equivalent second target Cₘᵢₙ of a 25 mg to 50 mg, a 25 mg to 300 mg or a 3 mg to 100 mg administration of a once daily immediate release oral composition of beta-3 adrenoceptor agonist. In embodiments, the pharmaceutical compositions reduce desensitization of beta-3 adrenoceptor, particularly when compared to an immediate release formulation of beta-3 adrenoceptor agonist that may be given, for example, once or twice daily. In embodiments, the pharmaceutical compositions achieve a plasma concentration [C] of beta-3 adrenoceptor agonist of about 1 μg/ml or below for a period of time of about 6 hours to about 9 hours during a 24 hour period. In embodiments, the pharmaceutical compositions achieve an AUC equivalent to the administration of about 25 mg to 50 mg, a 25 mg to 300 mg or a 3 mg to 100 mg of beta-3 adrenoceptor agonist in a once daily immediate release oral composition. In embodiments, the pharmaceutical composition achieves a target area under the curve (herein after AUC) over a 24 hour period.

In one embodiment the present application describes a once-daily treatment for LUTS that achieves a desired blood serum Cₘₐₓ while also not desensitizing the beta-3 adrenoceptor, comprising: a pharmaceutical composition, comprising a therapeutically effective amount of one or more beta-3 adrenoceptor agonists, wherein the pharmaceutical composition releases at least two pulses of one or more beta-3 adrenoceptor agonists, wherein a first pulse of one or more beta-3 adrenoceptor agonists achieves a first target Cₘₐₓ, a second pulse of one or more beta-3 adrenoceptor agonists achieves a second target Cₘₐₓ, a first target Cₘᵢₙ is achieved between the first pulse and the second pulse and a second Cₘᵢₙ is achieved after the second pulse. In embodiments, the first target Cₘₐₓ and the second target Cₘₐₓ are different. In embodiments, the first pulse and the second pulse are different amounts of the one or more beta-3 adrenoceptor agonists. Further embodiments describe treatments further comprising administering one or more additional therapeutic agents useful for the treatment of LUTS, wherein the one or more additional therapeutic agents are selected from the groups consisting of: antimuscarinic agents; alpha adrenoceptor blockers; 5-alpha reductases; and phosphodiesterase-5 inhibitors, wherein the one or more additional therapeutic agents may be administered prior to, simultaneously with, or following the administration of the pharmaceutical composition comprising one or more beta-3 adrenoceptor agonists. In embodiments, the beta-3 adrenoceptor agonist is selected from amibegron; mirabegron; ritobegron; vibegron; L-742,791; L-796,568; TRK-380, LY-368,842; Ro40-2148 and pharmaceutically acceptable salts thereof In embodiments, the beta-3 adrenoceptor agonist is selected from amibegron; mirabegron; ritobegron; and vibegron. In certain embodiments, the antimuscarinic agent is selected from the group consisting of tolterodine, oxybutynin, trospium, solifenacin, darifenacin, propiverine, fesoterodine, and pharmaceutically acceptable salts thereof.

In one embodiment the present application describes a once-daily treatment for LUTS that achieves a desired blood serum Cₘₐₓ while also not desensitizing the beta-3 adrenoceptor, comprising: at least one immediate release composition, comprising one or more beta-3 adrenoceptor agonists and at least one pharmaceutically acceptable carrier or diluent; and at least one modified release composition, comprising one or more beta-3 adrenoceptor agonists and at least one pharmaceutically acceptable carrier or diluent. Further embodiments describe treatments, further comprising administering one or more additional therapeutic agents useful for the treatment of LUTS, wherein the one or more additional therapeutic agents are selected from the groups consisting of: antimuscarinic agents; alpha adrenoceptor blockers; 5-alpha reductases; and phosphodiesterase-5 inhibitors, wherein the one or more additional therapeutic agents may be administered prior to, simultaneously with, or following the administration of the pharmaceutical composition comprising one or more beta-3 adrenoceptor agonists In embodiments, the beta-3 adrenoceptor agonist is selected from amibegron; mirabegron; ritobegron; vibegron; L-742,791; L-796,568; TRK-380, LY-368,842; and Ro40-2148 and pharmaceutically acceptable salts thereof. In embodiments, the beta-3 adrenoceptor agonist is selected from amibegron; mirabegron; ritobegron; and vibegron. In certain embodiments, the antimuscarinic agent is selected from the group consisting of tolterodine, oxybutynin, trospium, solifenacin, darifenacin, propiverine, fesoterodine, and pharmaceutically acceptable salts thereof

In one embodiment, the present application describes a dosing regimen for treating LUTS, comprising a.) administering a pharmaceutical composition comprising, a therapeutically effective amount of a beta-3 adrenoceptor agonist and at least one pharmaceutically acceptable carrier or diluent on days 1, 3, 5, 7, 9, 11, 13, 15, 17, 19. 21, 23, 25 and 27 of a 28 day dosing cycle; and b.) administering a pharmaceutical composition comprising, a therapeutically effective amount of anti-muscarinic agent and at least one pharmaceutically acceptable carrier or diluent on days 2, 4, 6, 8, 10, 12, 14, 16, 18, 20, 22, 24, 26 and 28 of a 28 day dosing cycle. In embodiments, the beta-3 adrenoceptor agonist is selected from amibegron; mirabegron; ritobegron; vibegron; solabegron; L-742,791; L-796,568; TRK-380; LY-368,842; Ro40-2148 and pharmaceutically acceptable salts thereof. In embodiments, the beta-3 adrenoceptor agonist is selected from amibegron; mirabegron; ritobegron; vibegron; L-742,791; L-796,568; TRK-380, LY-368,842; and Ro40-2148. In embodiments, the beta-3 adrenoceptor agonist is selected from amibegron; mirabegron; ritobegron; and vibegron. Further embodiments describe dosing regimens, wherein the anti-muscarinic agent is selected from the group consisting of: tolterodine; oxybutynin; trospium; solifenacin; darifenacin; propiverine; fesoterodine; and pharmaceutically acceptable salts thereof. Further embodiments describe dosing regimens, wherein the pharmaceutical composition comprising, a therapeutically effective amount of a beta-3 adrenoceptor agonist and at least one pharmaceutically acceptable carrier or diluent is an immediate-release composition. Further embodiments describe dosing regimens, wherein the pharmaceutical composition comprising, a therapeutically effective amount of a beta-3 adrenoceptor agonist and at least one pharmaceutically acceptable carrier or diluent is a modified-release composition. Further embodiments describe dosing regimens, wherein the pharmaceutical composition comprising a therapeutically effective amount of a beta-3 adrenoceptor agonist and at least one pharmaceutically acceptable carrier or diluent, comprises at least two pulses of the beta-3 adrenoceptor, wherein a first pulse of beta-3 adrenoceptor agonist achieves a first target Cₘₐₓ, a second pulse of beta-3 adrenoceptor agonist achieves a second target Cₘₐₓ, a first target Cₘᵢₙ is achieved between the first pulse and the second pulse and a second Cₘᵢₙ is achieved after the second pulse. Further embodiments describe dosing regimens, wherein the pharmaceutical composition comprising a therapeutically effective amount of an anti-muscarinic agent and at least one pharmaceutically acceptable carrier or diluent, is an immediate-release composition. Further embodiments describe dosing regimens, wherein the pharmaceutical composition comprising a therapeutically effective amount of an anti-muscarinic agent and at least one pharmaceutically acceptable carrier or diluent, is a modified-release composition. Further embodiments describe dosing regimens, wherein the pharmaceutical composition comprising a therapeutically effective amount of an anti-muscarinic agent and at least one pharmaceutically acceptable carrier or diluent, is administered q.o.d q.d.; b.i.d.; or t.i.d. Further embodiments describe dosing regimens, wherein the pharmaceutical composition comprising a therapeutically effective amount of a beta-3 adrenoceptor and at least one pharmaceutically acceptable carrier or diluent, is administered: q.o.d.; q.d.; b.i.d.; or t.i.d. In one embodiment, a dosing regimen comprises, administering a beta-3 adrenoceptor agonist and an antimuscarinic agent on alternating days.

In one embodiment, the present application describes a consumer pack for treating LUTS, comprising at least one beta-3 adrenoceptor agonist pharmaceutical composition, at least one of the anti-muscarinic pharmaceutical compositions described herein and a packet, comprising 28 separate containers arranged in a 28 day pattern, wherein the pharmaceutical composition comprising the beta-3 adrenoceptor and the pharmaceutical composition comprising the anti-muscarinic agent are in their appropriate containers. In one embodiment, the present application describes a consumer pack for treating LUTS, comprising at least one beta-3 adrenoceptor agonist pharmaceutical composition, and at least one of the anti-muscarinic agent pharmaceutical compositions described herein and a packet, comprising 28 separate containers, wherein the beta-3 adrenoceptor agonist is provided for on days 1, 3, 5, 7, 9, 11, 13, 15, 17, 19, 21, 23, 25, and 27and the anti-muscarinic agent is provided for on days 2, 4, 6, 8, 10, 12, 14, 16, 18, 20, 22, 24, 26, and 28. In embodiments, the beta-3 adrenoceptor agonist is selected from amibegron; mirabegron; ritobegron; vibegron; solabegron; L-742,791; L-796,568; TRK-380; LY-368,842; Ro40-2148 and pharmaceutically acceptable salts thereof. In embodiments, the beta-3 adrenoceptor agonist is selected from amibegron; mirabegron; ritobegron; vibegron; L-742,791; L-796,568; TRK-380, LY-368,842; and Ro40-2148. In embodiments, the beta-3 adrenoceptor agonist is selected from amibegron; mirabegron; ritobegron; and vibegron. In embodiments, the anti-muscarinic agent is selected from the group consisting of: tolterodine; oxybutynin; trospium; solifenacin; darifenacin; propiverine; fesoterodine; and pharmaceutically acceptable salts thereof.

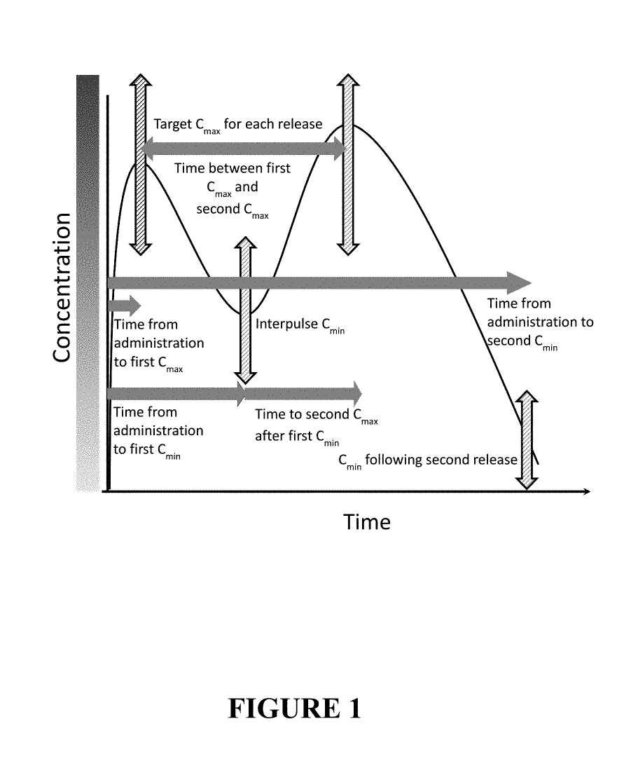

Embodiments of the present application describe pharmaceutical compositions comprising a therapeutically effective amount of beta-3 adrenoceptor agonist/mirabegron in a succession of at least two releases, wherein each release is optimized to provide a therapeutically effective plasma concentration [C] while also providing a lower plasma concentration [C] between the first and second release as well as between the second release and the subsequent administration of the pharmaceutical composition to allow for a sufficient recovery time for the beta-3 adrenoceptors and methods of using the same to treat diseases. An exemplary embodiment of such a pharmaceutical composition and its release profile is provided in FIG. 1.

In embodiments, the first release of beta-3 adrenoceptor agonist and the second release of beta-3 adrenoceptor agonist may be identical amounts or may be different amounts of beta-3 adrenoceptor agonist. In embodiments, the first release of beta-3 adrenoceptor agonist may be about 5 to 100 mg, 25 mg to 50 mg, 25 mg to 300 mg or a 3 mg to 100 mg. In embodiments, the first release of beta-3 adrenoceptor agonist may be about 20 to 80 mg. In embodiments, the first release of beta-3 adrenoceptor agonist may be about 30 to 70 mg. In embodiments, the first release may be about 40 to 60 mg.

In any of the foregoing embodiments, the immediate release composition and the modified release composition are separate and distinct pharmaceutical compositions. In any of the foregoing embodiments, the immediate release composition and the modified release composition are separate and distinct pharmaceutical compositions.

### Pharmaceutical Compositions

The pharmaceutical compositions of the present application can be administered orally or parenterally, such as subcutaneously or intravenously, as well as sublingually to various mammalian species known to be subject to such maladies, e.g., humans, in an effective amount up to about 1 gram, preferably up to about 800 mg, more preferably up to about 600 mg in a once-a-day regimen.

The pharmaceutical compositions of the present application can be administered for any of the uses described herein by any suitable means, for example, orally, such as in the form of tablets, capsules, granules or powders; sublingually; bucally; parenterally, such as by subcutaneous, intravenous, intramuscular, or intrasternal injection or infusion techniques (e.g., as sterile injectable aqueous or non-aqueous solutions or suspensions); in dosage unit formulations containing non-toxic, pharmaceutically acceptable vehicles or diluents. The present compositions can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compositions can also be administered liposomally.

The formulation for a beta-3 adrenoceptor agonist can significantly modify the absorption profile. For example, some compounds are differential absorbed in different regions of the GI tract. Some of the factors involved in absorption can include pH-dependent solubility, particle size, lipophilicity, ionization, GI-motility or transporters. Some pharmaceutical compositions are presented herein that improve the pH-dependent solubility of one or more beta-3 adrenoceptor agonists in the distal GI tract. Under these improved conditions, a second pulse of one or more beta-3 adrenoceptor agonists release and absorption will result. Additionally, methods for the release of one or more beta-3 adrenoceptor agonists in the distal GI tract based on pH are presented herein.

Another example of producing a delayed second pulse is based on the transit time of the dosage form. This is achievable through the time-dependent erosion of the dosage form coating. The GI transit time is well understood, and the coatings are designed to erode within a specific time range that corresponds to a specific region within the GI tract. Pharmaceutical compositions and methods of use are presented herein for the release of based on time-dependent erosion.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The compositions of the present application can also be delivered through the oral cavity by sublingual and/or buccal administration. Molded tablets, compressed tablets or freeze-dried tablets are exemplary forms which may be used. Exemplary compositions include those formulating the present beta-3 adrenoceptor agonists with fast dissolving diluents such as mannitol, lactose, sucrose and/or cyclodextrins. The compositions of the present application may take the form of pulsatile delivery systems such as, for example, PULSINCAP®, MICROPUMP®, MEDUSA™, PORT® system, CHRONOTROPIC®, TIME CLOCK®, multilayered tablets, DiffuCORE®, rupturable tablets, ACCU-BREAK® system, DIFFUCAPS®, DIFFUTABS®, Eurand MINITABS®, MICROCAPS®, SODAS®, IPDAS®, OsDrC®, OptiDose™, OptiMelt™, ZYDIS®, CODAS®, PRODAS®, TMDS®, DMDS®, PMDS®, GEOCLOCK®, GEOMATRIX®, PULSYS®, OROS® INTELLIMATRIX™ and VERSETROL™. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

The therapeutic agents in the pharmaceutical compositions of the present application may exist in any physical form known to one of skill in the art such as, for example, crystalline solids, amorphous solids, polymorphs, pharmaceutically acceptable salts, hydrates, solvates, stereoisomers, solutions and suspensions. Crystalline solids have regular ordered arrays of components held together by uniform intermolecular forces, whereas the components of amorphous solids are not arranged in regular arrays. Hydrates are substances that incorporate at least one water molecule into their crystalline matrix. Solvates are substances that incorporate at least one solvent molecule into their crystalline matrix. Polymorphs exhibit different crystalline structures for molecules that have the same molecular formula and sequence of bonded atoms. Stereoisomers are isomeric molecules that have the same molecular formula and sequence of bonded atoms (constitution), but that differ only in the three-dimensional orientations of their atoms in space.

Exemplary compositions for transdermal administration include transdermal therapeutic systems (hereinafter “TTS”). TTS are patches having a layered structure and comprising at least one active pharmaceutical ingredient in a reservoir layer. A distinction is made between matrix-type and reservoir-type TTS: in the first case the reservoir layer containing the active pharmaceutical ingredient has a pressure-sensitive adhesive finish, and in the second case a membrane which controls the rate of release of the active pharmaceutical ingredient, and where appropriate an additional pressure-sensitive adhesive layer, are present.

Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

In embodiments, beta-3 adrenoceptor agonist is beta-3 adrenoceptor agonist or a pharmaceutically acceptable salt thereof In embodiments, beta-3 adrenoceptor agonist is amorphous or the free base. In embodiments, mirabegron is mirabegron or a pharmaceutically acceptable salt thereof In embodiments, solabegron is solabegron or a pharmaceutically acceptable salt thereof In embodiments, mirabegron is amorphous or the free base. In embodiments, solabegron is amorphous, the free base or zwitterion. In embodiments, a pharmaceutically acceptable salt thereof may include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)), various amino acids, aluminum, calcium, lithium, magnesium, potassium, sodium, zinc, iron, diethanolamine, amines, such as organic amines, N,N′-dibenzylethylenediamine, chloroprocaine, choline, diethanolamine, dicyclohexylamine, ethylenediamine, N-methylglucamine, and procaine. In embodiments, mirabegron is mirabegron hydrochloride. In embodiments solabegron is solabegron hydrochloride

It will be understood that the specific dose level and frequency of dosage for any particular subject can be varied and will depend upon a variety of factors including the activity of the specific beta-3 adrenoceptor agonist employed, the metabolic stability and length of action of that compound, the species, age, body weight, general health, sex and diet of the subject, the mode and time of administration, rate of excretion, drug combination, and severity of the particular condition.

In embodiments, the first release of beta-3 adrenoceptor agonist may be a pulsatile release of beta-3 adrenoceptor agonist. In embodiments, the second release of beta-3 adrenoceptor agonist may be a pulsatile release of beta-3 adrenoceptor agonist. In embodiments, the first release of beta-3 adrenoceptor agonist may be an immediate release of beta-3 adrenoceptor agonist. In embodiments, the second release of beta-3 adrenoceptor agonist may be an immediate release of beta-3 adrenoceptor agonist. In embodiments, the first release of beta-3 adrenoceptor agonist may be modified release of beta-3 adrenoceptor agonist. In embodiments, the second release of beta-3 adrenoceptor agonist may be a modified release of beta-3 adrenoceptor agonist. In embodiments, the first release of beta-3 adrenoceptor agonist may be an extended release of beta-3 adrenoceptor agonist. In embodiments, the second release of beta-3 adrenoceptor agonist may be an extended release of beta-3 adrenoceptor agonist. In embodiments, the first release of beta-3 adrenoceptor agonist may be a delayed release of beta-3 adrenoceptor agonist. In embodiments, the second release of beta-3 adrenoceptor agonist may be a delayed release of beta-3 adrenoceptor agonist. In embodiments, the first release of beta-3 adrenoceptor agonist may be a multiparticulate formulation of beta-3 adrenoceptor agonist. In embodiments, the second release of beta-3 adrenoceptor agonist may be a multiparticulate formulation of beta-3 adrenoceptor agonist. In embodiments, the first release of beta-3 adrenoceptor agonist may be a matrix formulation of beta-3 adrenoceptor agonist. In embodiments, the second release of beta-3 adrenoceptor agonist may be a matrix formulation of beta-3 adrenoceptor agonist. In embodiments, the first and second release of beta-3 adrenoceptor agonist may be any combination of the foregoing.

In embodiments, the pharmaceutical composition may be a multiparticulate formulation. In embodiments, the multiparticulate formulation may comprise at least two populations of pellets containing beta-3 adrenoceptor agonist. In embodiments, a first population of pellets is immediate release and a second population is delayed, sustained or modified release. In embodiments, the first population of pellets release the beta-3 adrenoceptor agonist immediately in the upper GI tract and the second population of pellets release the beta-3 adrenoceptor agonist later in a lower portion of the GI tract. In embodiments, the second population of pellets that are delayed, sustained or modified release may be coated with a pH dependent coating or a time dependent coating so as to delay the second release of beta-3 adrenoceptor agonist to the desired position in the GI tract. In embodiments, the pellets may be drug-layered and/or matrix-type pellets.

In embodiments, the pharmaceutical composition may be a drug-coated sphere(s) formulation. In embodiments, the formulation may comprise at least two populations of drug-coated spheres containing beta-3 adrenoceptor agonist. In embodiments, a first population of drug-coated spheres release the beta-3 adrenoceptor agonist immediately in the upper GI tract and the second population of drug-coated spheres release the beta-3 adrenoceptor agonist later in a lower portion of the GI tract. In embodiments, the second population of drug-coated spheres may be coated with a pH dependent coating or a time dependent coating so as to delay the second release of beta-3 adrenoceptor agonist to the desired position in the GI tract.

In embodiments, the pharmaceutical composition may be a bi-layer tablet or a dual-encapsulated capsule. In embodiments, the bi-layer tablet may comprise an immediate release layer and a delayed, sustained or modified release layer. The immediate layer may release beta-3 adrenoceptor agonist immediately in the GI tract and the modified, delayed or sustained release layer will release beta-3 adrenoceptor agonist at a later time and lower in the GI tract. The modified release layer may be coated with either a pH dependent coating or a time dependent coating so as to delay the second release of beta-3 adrenoceptor agonist to the desired position in the GI tract.

In embodiments, the pharmaceutical composition may be a matrix tablet. In embodiments, the matrix tablet may comprise a well-mixed composite of drug(s) with rate-controlling excipients. Numerous sustained and/or delayed release tablets such as membrane controlled system, matrices with water soluble/insoluble polymers, and osmotic systems may be utilized. The tablet may contain either the amorphous form of beta-3 adrenoceptor agonist or the crystalline form. The delayed/sustained release can be achieved by applying a permeable or semipermeable membrane to the tablet core or by mixing the drug with excipient that is either a hydrophilic polymer with high viscosity and gel forming capability or a hydrophobic excipient that slows down the diffusion of drug molecule. An immediate release drug layer can be coated to the tablet that will be available for an early release in the GI tract, while the delayed release core will be designed to delay the drug release after a time period in a designed region of the GI tract.

In embodiments, the pharmaceutical composition may be a multicore tablet. In embodiments the multicore tablet may comprise multiple discrete cores consisting of at least one immediate release core and at least one delayed/sustained release core contained within the same tablet. The at least one immediate release core will be available for an early release in the GI tract, while the at least one delayed/sustained release core will be designed to delay the drug release after a time period in a designed region of the GI tract.

In embodiments, the pharmaceutical composition may be a gastroretentive oral delivery system. In embodiments the gastroretentive oral delivery system may comprise a gastroretentive oral dosage form containing beta-3 adrenoceptor agonist for the multiple releases of beta-3 adrenoceptor agonist to a patient in need. The formulation will contain a tablet or capsule having both an immediate release and modified release component. The immediate layer will release beta-3 adrenoceptor agonist immediately in the GI tract , wherein the modified release layer will release beta-3 adrenoceptor agonist at a later time inside the GI tract. The gastroretentive oral dosage form may utilize mucoadhesive, swellable, high density or floating technologies to prolong residence time in the stomach thereby allowing a prolonged period for release of both first and second releases in the stomach or upper GI. Both releases may contain any physical form of beta-3 adrenoceptor agonist such as, for example, amorphous or crystalline solid.

### Pharmaceutical Combinations

The present application includes within its scope pharmaceutical compositions comprising, as an active ingredient, a therapeutically effective amount of one or more beta-3 adrenoceptor agonists, alone or in combination with a pharmaceutical carrier or diluent. Optionally, the pharmaceutical compositions of the present invention can be used alone, or in combination with other suitable therapeutic agents useful in the treatment of the LUTS including: antimuscarinic agents, alpha adrenoceptor blockers, 5-alpha reductases and phosphodiesterase-5 inhibitors.

In embodiments, the pharmaceutical composition may further comprise a therapeutically effective amount of one or more additional therapeutic agents. In embodiments, the one or more additional therapeutic agents may be an antimuscarinic agents, alpha adrenoceptor blockers, botulinum toxin, purinergics, cannabinoids, transient receptor potential (TRP) protein inhibitors, prostaglandins, 5-alpha reductase inhibitors, phosphodiesterase-5 inhibitors or percutaneous tibial nerve stimulation. In embodiments, the antimuscarinic agent may be tolterodine, oxybutynin, trospium, solifenacin, darifenacin, propiverine, fesoterodine, and pharmaceutically acceptable salts thereof. In embodiments, alpha adrenoceptor blockers may be tamuslosin, alfuzosin, and silodosin and pharmaceutically acceptable salts thereof In embodiments, 5-alpha reductase inhibitors may be finasteride, dutaseteride and pharmaceutically acceptable salts thereof. In embodiments, phosphodiesterase-5 inhibitors may be sildenafil, tadalafil, vardenafil, udenafil, avanafil and pharmaceutically acceptable salts thereof

Such other therapeutic agent(s) may be administered prior to, simultaneously with, or following the administration of the beta-3 adrenoceptor agonist containing pharmaceutical composition in accordance with the invention.

Examples of suitable antimuscarinic agents for use in combination with the pharmaceutical compositions of the present application include tolterodine, oxybutynin, trospium, solifenacin, darifenacin, propiverine, fesoterodine, and pharmaceutically acceptable salts thereof

Examples of suitable alpha adrenoceptor blockers for use in combination with the pharmaceutical compositions of the present application include tamuslosin, alfuzosin, and silodosin.

Examples of suitable 5-alpha reductases for use in combination with the pharmaceutical compositions of the present application include finasteride, dutaseteride and pharmaceutically acceptable salts thereof

Examples of suitable phosphodiesterase-5 inhibitors for use in combination with the pharmaceutical compositions of the present application include sildenafil, tadalafil, vardenafil, udenafil, avanafil and pharmaceutically acceptable salts thereof.

### Methods of Treatment

In one embodiment the present application describes a method of treating one or more symptoms of over active bladder, comprising: administering a pharmaceutical composition, comprising a therapeutically effective amount of one or more beta-3 adrenoceptor agonists and at least one pharmaceutically acceptable diluent or carrier, wherein the one or more symptoms of overactive bladder are selected from the group consisting of: increased urinary urgency of urination, increase in urinary micturition frequency, urinary incontinence, painful urination, excessive passage of urine at night, poor stream, overactive bladder, hesitancy, terminal dribbling, incomplete voiding and overflow incontinence. Further embodiments describes methods, wherein the pharmaceutical composition will be administered in the morning or the pharmaceutical composition will be administered with a meal In embodiments, the beta-3 adrenoceptor agonist is selected from amibegron; mirabegron; ritobegron; vibegron; solabegron; L-742,791; L-796,568; TRK-380; LY-368,842; Ro40-2148 and pharmaceutically acceptable salts thereof. In embodiments, the beta-3 adrenoceptor agonist is selected from amibegron; mirabegron; ritobegron; vibegron; L-742,791; L-796,568; TRK-380, LY-368,842; and Ro40-2148. In embodiments, the beta-3 adrenoceptor agonist is selected from amibegron; mirabegron; ritobegron; and vibegron.

In one embodiment the present application describes a method for treating LUTS, comprising: administering a pharmaceutical composition, comprising a therapeutically effective amount of one or more beta-3 adrenoceptor agonists, wherein the pharmaceutical composition releases at least two pulses of one or more beta-3 adrenoceptor agonists, wherein a first pulse of one or more beta-3 adrenoceptor agonists achieves a first target Cₘₐₓ, a second pulse of one or more beta-3 adrenoceptor agonists achieves a second target Cₘₐₓ, a first target Cₘᵢₙ is achieved between the first pulse and the second pulse and a second Cₘᵢₙ is achieved after the second pulse. In embodiments, the first target Cₘₐₓ and the second target Cₘₐₓ are different. In embodiments, the first pulse and the second pulse are different amounts of the one or more beta-3 adrenoceptor agonists. Further embodiments describes methods that further comprise administering one or more additional therapeutic agents useful for the treatment of LUTS, wherein the one or more additional therapeutic agents are selected from the groups consisting of: antimuscarinic agents; alpha adrenoceptor blockers; 5-alpha reductases; and phosphodiesterase-5 inhibitors, wherein the one or more additional therapeutic agents may be administered prior to, simultaneously with, or following the administration of the pharmaceutical composition comprising one or more beta-3 adrenoceptor agonists. Still further embodiments describe methods, wherein the pharmaceutical composition is administered every other day (QOD); once a day (QD), twice a day (BID) or three times a day (TID) to a patient in need thereof In embodiments, the beta-3 adrenoceptor agonist is selected from amibegron; mirabegron; ritobegron; vibegron; L-742,791; L-796,568; TRK-380, LY-368,842; and Ro40-2148. In embodiments, the beta-3 adrenoceptor agonist is selected from amibegron; mirabegron; ritobegron; and vibegron. In certain embodiments, the antimuscarinic agent is selected from the group consisting of tolterodine, oxybutynin, trospium, solifenacin, darifenacin, propiverine, fesoterodine, and pharmaceutically acceptable salts thereof. In some embodiments, the target Cₘₐₓ achieves between about 50% and about 100% of the beta-3 adrenoceptor response as measured by urinary urgency, frequent urination, nocturia, urinating unintentionally, urge incontinence or bladder capacity. In some embodiments, the target Cₘᵢₙ achieves between about 50% and about 0% of the beta-3 adrenoceptor response as measured by urinary urgency, frequent urination, nocturia, urinating unintentionally, urge incontinence or bladder capacity. In some embodiments, the first pulse comprises about 12 mg to about 200 mg of one or more beta-3 adrenoceptor agonists. In some embodiments, the second pulse comprises about 12 mg to about 200 mg of one or more beta-3 adrenoceptor agonists. In embodiments, the pharmaceutical composition reduces desensitization of beta-3 adrenoceptor or otherwise increases the therapeutic effect of beta-3 adrenoceptor agonist, particularly when compared to an immediate release formulation of beta-3 adrenoceptor agonist that may be given, for examples, twice daily. Desensitization occurs when the beta-3 adrenoceptor is not otherwise responsive to an agonist, is less responsive to an agonist, or the target tissue (e.g., the bladder) is not otherwise responsive or is less responsive to an agonist. In embodiments, the first target Cₘₐₓ is less than the equivalent Cₘₐₓ of a 25 mg to 50 mg, a 25 mg to 300 mg or a 3 mg to 100 mg administration of a once daily, immediate release oral composition of beta-3 adrenoceptor agonist. In embodiments, the first target Cₘᵢₙ is less than the equivalent Cₘᵢₙ of a 25 mg to 50 mg, a 25 mg to 300 mg or a 3 mg to 100 mg administration of a once daily, immediate release oral composition of beta-3 adrenoceptor agonist. In embodiments, the second target Cₘₐₓ is less than the equivalent second target Cₘₐₓ of a 25 mg to 50 mg, a 25 mg to 300 mg or a 3 mg to 100 mg administration of a once daily immediate release oral composition of beta-3 adrenoceptor agonist. In embodiments, the second target Cₘᵢₙ is less than the equivalent second target Cₘᵢₙ of a 25 mg to 50 mg, a 25 mg to 300 mg or a 3 mg to 100 mg administration of a once daily immediate release oral composition of beta-3 adrenoceptor agonist. In embodiments, the pharmaceutical compositions reduce desensitization of beta-3 adrenoceptor, particularly when compared to an immediate release formulation of beta-3 adrenoceptor agonist that may be given, for example, once or twice daily. In embodiments, the pharmaceutical compositions achieve a plasma concentration [C] of beta-3 adrenoceptor agonist of about 1 μg/ml or below for a period of time of about 6 hours to about 9 hours during a 24 hour period. In embodiments, the pharmaceutical compositions achieve an AUC equivalent to the administration of about 25 mg to 50 mg, a 25 mg to 300 mg or a 3 mg to 100 mg of beta-3 adrenoceptor agonist in a once daily immediate release oral composition. In embodiments, the pharmaceutical composition achieves a target area under the curve (herein after AUC) over a 24 hour period.

In one embodiment the present application describes a method for treating LUTS, comprising: administering a pharmaceutical composition for the delivery of one or more beta-3 adrenoceptor agonists, comprising: an immediate release composition, comprising one or more beta-3 adrenoceptor agonists and at least one pharmaceutically acceptable carrier or diluent; and a modified release composition, comprising one or more beta-3 adrenoceptor agonists and at least one pharmaceutically acceptable carrier or diluent to a patient in need thereof. Additional embodiments describe methods, wherein the pharmaceutical composition is administered every other day (QOD); once a day (QD), twice a day (BID) or three times a day (TID) to a patient in need thereof. Further embodiments describe methods, further comprising administering one or more additional therapeutic agents useful for the treatment of LUTS, wherein the one or more additional therapeutic agents are selected from the groups consisting of: antimuscarinic agents; alpha adrenoceptor blockers; 5-alpha reductases; and phosphodiesterase-5 inhibitors, wherein the one or more additional therapeutic agents may be administered prior to, simultaneously with, or following the administration of the pharmaceutical composition comprising one or more beta-3 adrenoceptor agonists In embodiments, the beta-3 adrenoceptor agonist is selected from amibegron; mirabegron; ritobegron; vibegron; L-742,791; L-796,568; TRK-380, LY-368,842; Ro40-2148 and pharmaceutically acceptable salts thereof. In embodiments, the beta-3 adrenoceptor agonist is selected from amibegron; mirabegron; ritobegron; and vibegron. In certain embodiments, the antimuscarinic agent is selected from the group consisting of tolterodine, oxybutynin, trospium, solifenacin, darifenacin, propiverine, fesoterodine, and pharmaceutically acceptable salts thereof In some embodiments, the at least one immediate release composition comprises about 12 mg to about 200 mg of one or more beta-3 adrenoceptor agonists. In some embodiments, the at least one modified release composition comprises about 12 mg to about 200 mg of one or more beta-3 adrenoceptor agonists.

In one embodiment, the present application describes a method for treating LUTS utilizing any one of the dosing regimens described herein. In one embodiment, a method of treating LUTS comprises administering a beta-3 adrenoceptor and an antimuscarinic agent on alternating days In embodiments, the method of treating LUTS, comprising a.) administering a pharmaceutical composition comprising, a therapeutically effective amount of a beta-3 adrenoceptor agonist and at least one pharmaceutically acceptable carrier or diluent on days 1, 3, 5, 7, 9, 11, 13, 15, 17, 19, 21, 23, 25 and 27 of a 28 day dosing cycle; and b.) administering a pharmaceutical composition comprising, a therapeutically effective amount of anti-muscarinic agent and at least one pharmaceutically acceptable carrier or diluent on days 2, 4, 6, 8, 10, 12, 14, 16, 18, 20, 22, 24, 26 and 28 of a 28 day dosing cycle. In embodiments, the beta-3 adrenoceptor agonist is selected from amibegron; mirabegron; ritobegron; vibegron; solabegron; L-742,791; L-796,568; TRK-380; LY-368,842; Ro40-2148 and pharmaceutically acceptable salts thereof In embodiments, the beta-3 adrenoceptor agonist is selected from amibegron; mirabegron; ritobegron; vibegron; L-742,791; L-796,568; TRK-380, LY-368,842; and Ro40-2148. In embodiments, the beta-3 adrenoceptor agonist is selected from amibegron; mirabegron; ritobegron; and vibegron. Further embodiments describe methods, wherein the anti-muscarinic agent is selected from the group consisting of: tolterodine; oxybutynin; trospium; solifenacin; darifenacin; propiverine; fesoterodine; and pharmaceutically acceptable salts thereof. Further embodiments describe methods, wherein the pharmaceutical composition comprising, a therapeutically effective amount of a beta-3 adrenoceptor agonist and at least one pharmaceutically acceptable carrier or diluent is an immediate-release composition. Further embodiments describe methods, wherein the pharmaceutical composition comprising, a therapeutically effective amount of a beta-3 adrenoceptor agonist and at least one pharmaceutically acceptable carrier or diluent is a modified-release composition. Further embodiments describe methods, wherein the pharmaceutical composition comprising a therapeutically effective amount of a beta-3 adrenoceptor agonist and at least one pharmaceutically acceptable carrier or diluent, comprises at least two pulses of the beta-3 adrenoceptor, wherein a first pulse of beta-3 adrenoceptor agonist achieves a first target Cₘₐₓ, a second pulse of beta-3 adrenoceptor agonist achieves a second target Cₘₐₓ, a first target Cₘᵢₙ is achieved between the first pulse and the second pulse and a second Cₘᵢₙ is achieved after the second pulse. Further embodiments describe methods, wherein the pharmaceutical composition comprising a therapeutically effective amount of an anti-muscarinic agent and at least one pharmaceutically acceptable carrier or diluent, is an immediate-release composition. Further embodiments describe methods, wherein the pharmaceutical composition comprising a therapeutically effective amount of an anti-muscarinic agent and at least one pharmaceutically acceptable carrier or diluent, is a modified-release composition. Further embodiments describe methods, wherein the pharmaceutical composition comprising a therapeutically effective amount of an anti-muscarinic agent and at least one pharmaceutically acceptable carrier or diluent, is administered q.o.d q.d.; b.i.d.; or t.i.d. Further embodiments describe methods, wherein the pharmaceutical composition comprising a therapeutically effective amount of a beta-3 adrenoceptor and at least one pharmaceutically acceptable carrier or diluent, is administered: q.o.d.; q.d.; b .d.; or t.i.d.

In embodiments, methods of treating a disease comprising administering to a subject in need thereof a pharmaceutical composition as described herein are provided. In embodiments, the disease may be LUTS, obesity, type 2 diabetes, heart failure, irritable bowel syndrome and similar gastrointestinal disorders, pre-term labor, depression anxiety, and combinations thereof. In embodiments, treating LUTS may include treating or otherwise decreasing frequency of urgency, decreasing nocturia, decreasing urinary micturition frequency, decreasing urinary incontinence, increasing voided volume, decreasing post-void residual volume, and/or improving patient reporting outcomes.

In embodiments, methods of treating such diseases may further comprise administering a therapeutically effective amount of one or more additional therapeutic agents. In embodiments, the one or more additional therapeutic agents may be administered prior to, simultaneously with, or following the administration of the pharmaceutical composition comprising a beta-3 adrenoceptor agonist. In embodiments, the one or more additional therapeutic agents may be an antimuscarinic agents, alpha adrenoceptor blockers, botulinum toxin, purinergics, cannabinoids, transient receptor potential (TRP) protein inhibitors, prostaglandins, 5-alpha reductase inhibitors, phosphodiesterase-5 inhibitors or percutaneous tibial nerve stimulation. In embodiments, the antimuscarinic agent may be tolterodine, oxybutynin, trospium, solifenacin, darifenacin, propiverine, fesoterodine, and pharmaceutically acceptable salts thereof

In any of the foregoing embodiments, the immediate release composition and the modified release composition are separate and distinct pharmaceutical compositions. In any of the foregoing embodiments, the immediate release composition and the modified release composition are separate and distinct pharmaceutical compositions.

### Examples

HEK cells transfected with the human beta-3 adrenoceptor according to the method of Vrygag et al (2009) will be employed. Additional cell lines, such as CHO, SK-N-MC neuroblastoma cells or cultured human adipocytes may be considered.

For the desensitization experiments, the cells will be cultured for 0.5 hr to 24 hr in a serum-free medium in the presence of vehicle or 10-μM beta-3 agonists. Beta -3 agonists that may be studied include solabegron, mirabegron, CL 316,243 or isoproterenol. Thereafter, cells will be thoroughly in serum-free medium.

### Example 1

### Cyclic AMP Endpoint Assessment of Beta-3 Adrenoceceptor Desensitization

Cells will be detached from the surface using enzyme-free cell dissociation buffer and washed once with Hank's balanced salt solution (HBSS). Cell will be re-suspended in HBSS supplemented with 5 mM HEPES and 0.05% bovine serum albumin. The cells will be stimulated with the appropriate beta-3 agonists. The stimulation mixture will contain the cAMP phosphodiesterase inhibitors IBMX and RO 20-1724 (100 μM each). Cells will be added to the stimulation mixture 1:1 in a 384 well optiplate and stimulated for 30 min at room temperature. cAMP detection will be suing a LANCE® cAMP Kit (PerkinElmer).

### Example 2

### Radioligand-binding Studies Endpoint Assessment of Beta-3 Adrenocepeceptor Desensitization

[³H]-L 748,337 saturation radioligand binding will be performed as previously described (van Wieringen et al. 2011). Briefly, cells at approximately 80% confluence will be washed with PBS, harvested by scraping the culture flasks with a cell scraper, washed twice by centrifugation, and then homogenized in ice-cold buffer (50 mM Tris, 0.5 mM EDTA, pH 7.5). The homogenates will be centrifuged for 20 min at 50,000×g at 4° Cₘₐₓ The pellets will be resuspended in buffer and stored at −80 ° C. Aliquots of the respective membrane preparation (approximately 50-100 μg protein/assay) will be incubated in a total volume of 250 μl of binding buffer (10 mM Tris, 0.9 mN NaCl at pH 7.4) at 25° C. for 60 min. Non-specific binding will be defined using 100 μM isoproterenol. In saturation experiments, eight radioligand concentrations will be used. All experiments will be performed in duplicates in 96 well plates, and incubations will be terminated by rapid vacuum filtration. Each filter will be washed with approximately 2-3 ml of ice-cold buffer. Radioactivity adherent to the filters will be quantified in Perkin Elmer scintillator counter.

### Example 3

### Alteration in G-Protein Expression Endpoint Assessment of Beta-3 Receptor Desensitization

Cells treated with beta-3 agonists at various time-points will be washed with PBS, harvested, homogenized and centrifuged. The pellets will be re-homogenized, boiled, loaded onto SDS gels and electrophoresed for approximately 1 hr at 40 mA. Primary antibodies (rabbit polyclonal) for detection of G protein subunits (GSK, Gi1,Gi2, Gi3,Gq/11) will be used. Immunoblotting will be performed for approximately 12 hr at 4° C. Following washing, a secondary antibody (i.e. donkey anti-rabbit coupled to horseradish peroxidase) will be used. Luminescence signals will be detected and quantified.

### Example 4

### Prevention of Receptor Desensitization

In these experiments, the cells in which the beta-3 adrenoceptor has been desensitized due to prolonged exposure of a beta-3 agonists (described above) will be employed. Cells that have desensitized beta-3 adrenoceptors will be washed out with culture medium buffer for a pre-specified amount of time. This time range will be from 0.5 hr to 24 hr. Following this wash out period, the cells will be re-exposed to the beta-3 agonist for a pre-specified time period. This time-range will be from 0.5 to 24 hr. The optimum time-range for washout and beta-3 agonist re-exposure will be determined experimentally. Following the second exposure of the beta-3 agonist will be washed with serum-free medium and the endpoints described above will be assessed.

### Example 5

### Isometric-Tension Recording in Organ Bath Experiments

Rat bladder specimens will be Wistar rats (body weight 190-260 g). All animals will be group-housed in cages at least 4-5 days before the experiments with free access to food and water. Rats will be humanely euthanized using CO₂. The whole urinary bladder will be isolated, freed from connective and fat tissues, the dome and base removed and the bladder bisected into two halves (strips). The urothelium will be removed.

Human bladder specimens will be obtained perioperatively from patients undergoing cystectomy due to bladder cancer. Tissues were placed in a cold storage solution and transported to the laboratory immediately after surgery in a container at 4° C. Upon receipt, tissues will be stored at 4° C. until the start of the experiment. After removal of the serosa and mucosa, 8-10 mm long×2-3 mm wide detrusor smooth muscle strips will be prepared. The urothelium will be removed.

Both human and rat strips will be mounted in glass organ bath chambers containing Krebs-Henseleit solution (pH 7.4) and gassed with 95% O₂ and 5% CO₂ at 37° C. Tissues will be allowed to equilibrate under a resting tension of 1 g for 60-90 min. Strips will be then exposed to KCl (80 mM) to measure their viability. Bladder responses will be measured using isometric transducers and recorded using a data acquisition system.

The effect of a beta-3 adrenoceptor agonists on contractions evoked by exogenous carbachol or KCl will be assessed. Continuous active tone will be induced by carbachol (0.1-1 uM) KCl (40-80 mM). Concentration-dependent relaxation with appropriate beta-3 adrenoceptor agonists will be performed by be adding the beta-3 agonist to the organ bath chamber at 5-10 min. intervals or until the plateau of the relaxation response is achieved. When maximal relaxation is obtained the tissues will then be further incubated with maximal concentration of the beta-3 agonists (i.e., 10 uM) for a specified amount of time. This amount of time will be assessed between 0.25 to 24 hr (i.e., 0.25, 0.5, 1, 3, 6,12, 24 hr). In some experiments the beta-3 receptor antagonist SR59230A may be used to confirm the response is beta-3 adrenoceptor-mediated.

Following the incubation of the bladder tissue with the beta-3 adrenoceptor agonists, the organ bath chamber will be rinsed with Krebs buffer for a specified period of time. Again, this amount of time will be assessed between 0.25 to 24 hr. (i.e., 0.25, 0.5, 1, 3, 6, 12, 24 hr). Following the wash-out period the bladder tissues will be re-contracted with carbachol and concentration-dependent relaxation studies will be repeated.

Tissue responses will be calculated as the mean (SEM) and expressed as a percentage of the active induced tone (i.e., carbachol), or as a percentage of the control response.

### Example 6

### Prevention of Receptor Desensitization

Macroscopically normal bladders from Male CD rats (220-250 g) were used. Furthermore, tissues were rejected if they did not respond adequately to viability checks. Each bladder was cleaned free of surrounding connective tissue and halved longitudinally. The bladder longitudinal smooth muscle, with the urothelium still attached were mounted in 25 ml organ baths containing physiological saline solution (PSS) (composition: 119.0 mM NaCl, 4.7 mM KCl, 1.2 mM MgSO₄, 24.9 mM NaHCO₃, 1.2 mM KH₂PO₄, 2.5 mM CaCl₂ and 11.1 mM glucose) supplemented with 1 μM prazosin (α₁-adrenoceptor antagonist) and 30 nM ICI118551 (β₂-adrenoceptor antagonist), aerated with 95% O₂/5% CO₂ gas mix, warmed and maintained at approximately 37° C. throughout the experiment. Changes in force production were recorded using transducers. After mounting, the bladder halves were allowed to equilibrate for at least 30 min before they were set to a stable tension of approximately 1.0 g. The tissue was then allowed to equilibrate over at least a 60 min period with washes every 15 min.

EFS parameters and viability check: In initial experiments the parameters for the EFS were assessed by performing a frequency curve to determine a frequency that would give a response that was approximately 80% of the response seen to 80 mM KCl. Optimal EFS parameters were determined to be: 30 Volts, square pulse of 0.1 ms, train of 4 seconds every 120 seconds, 15 Hz. This frequency was then used to stimulate the tissue for all subsequent experiments. The viability of bladder strips was tested by stimulating the tissue with EFS for minimum of ten min. Tissues that failed to produce a response of at least 1.0 g were rejected.

Pilot studies to determine EC₉₀ concentrations of the test compounds were performed. Upon stabilization of the baseline tension, the bladder muscle strips were stimulated with EFS parameters described above. The resulting contractile responses were allowed to stabilize before adding cumulative concentrations of test compound (half-log increments) in a cumulative concentration response curve (CCRC). A vehicle and a positive control (CL-316,243) were run in the same manner in order to compare with the test compounds. From the data obtained in these experiments, EC₉₀ concentrations of the test compounds were determined for use in subsequent experiments.

The bladder muscle strips were incubated with the EC₉₀ determined for each test compound in the pilot studies, for a period of 1 or 3 hr. Following compound incubation, the tissues were washed with PSS for a period of 1, 3 or 6 hr, with washes approximately every 15 min, to remove the drug. At the end of the final wash period the tissues were stimulated with EFS, and left to equilibrate for at least 30 min. -A CCRC was then performed in each tissue.

Determination of EC₉₀ concentration for beta-3 adrenoceptor agonists to inhibit EFS-induced contraction in rat isolated bladder: In the initial pilot experiments, concentration response curves were performed to the test compounds to determine an EC₉₀ value for use in later experiments. The calculated values for CL-316,243 and mirabegron were 0.042, and 15.3 respectively. Effect of the washout period following a one hr incubation with test compound.

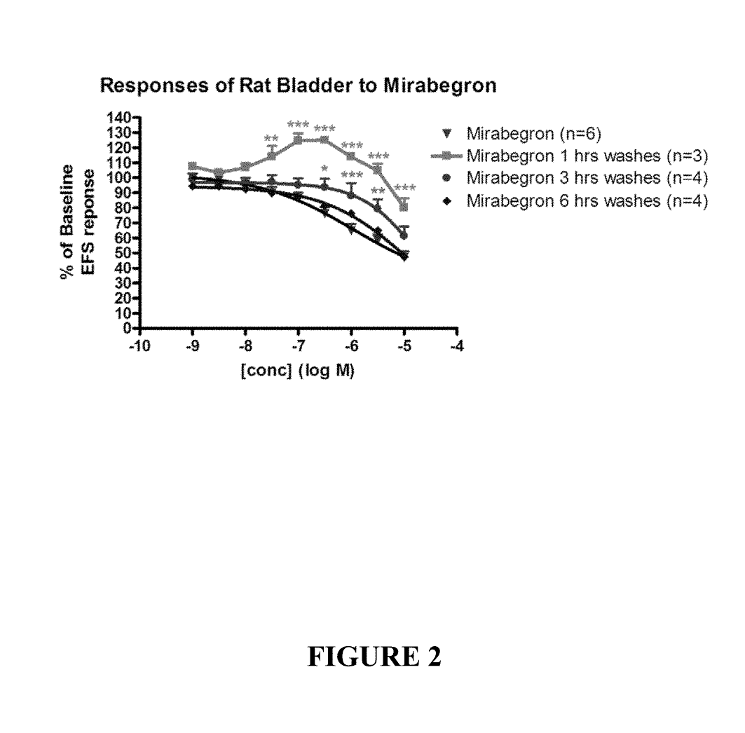

Effect of the washout period following a one hr incubation with test compound: Tissues were incubated with the EC₉₀ concentration of the test compounds for one hr followed by one hr, three hr or six hr of washing with PSS. After only one hr of washing, the responses to mirabegron were significantly attenuated (FIG. 2). Responses to higher concentrations of mirabegron were also significantly attenuated after three hr of washing. After six hr of washing the responses to mirabegron were similar to that seen in tissues that had not been pre-exposed to the test compound (FIG. 2). Mirabegron produced marked potentiation of EFS-induced contraction from baseline at the lower and mid-concentration range. These data suggest that incubation of the rat bladder with beta-3 adrenoceptor agonists produced marked receptor desensitization, and the receptor is re-sensitized in a time-dependent manner following removal of the agonist by removing the ligand by washing out.

Effect of the washout period following three hr incubation with test compound: In the next series of experiments, tissues were incubated with the EC₉₀ concentration of the test compounds for three hr followed by either one hr, three hr or six hr of washing with PSS.

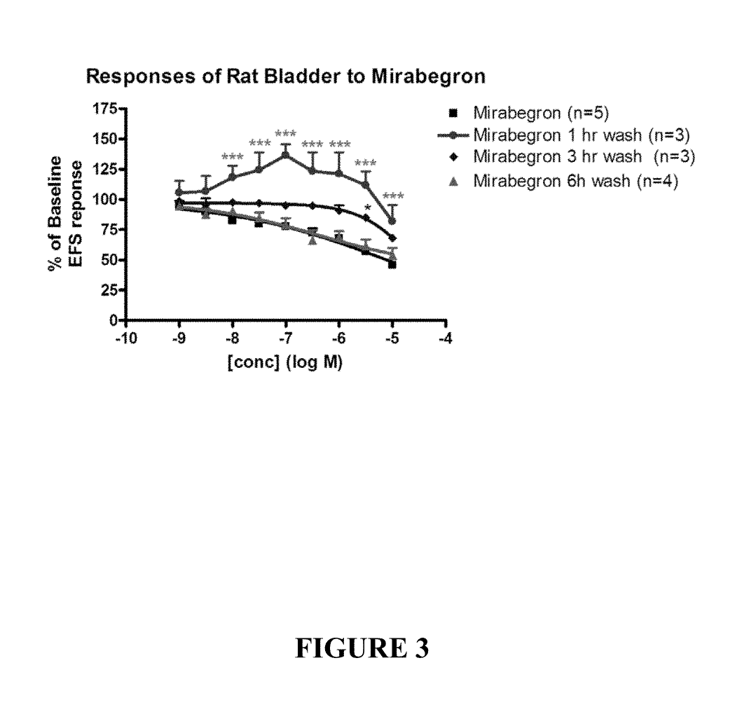

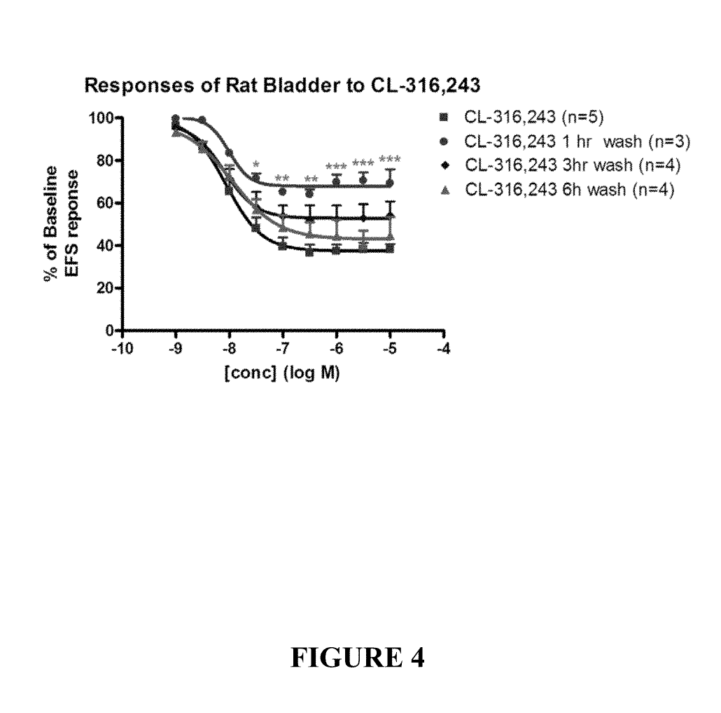

After only one hr of washing, the responses to mirabegron and CL-316,243 were significantly attenuated (FIGS. 3 AND 4, respectively). Responses to the highest concentrations of mirabegron were also significantly attenuated after three hr of washing. After six hr of washing the responses to mirabegron or to CL-316,243 were similar to that seen in tissues which had not been pre-exposed to the test compound (FIGS. 3 AND 4, respectively).

As observed with the one hr incubation data, unlike CL316,243, mirabegron produced marked potentiation of EFS-induced contraction from baseline at the low and mid-concentration range. This EFS-induced potentiation of bladder contraction was not observed with CL 316,243. These data suggest that incubation of the rat bladder with beta-3 adrenoceptor agonists produced marked receptor desensitization, and the receptor is re-sensitized in a time-dependent manner following removal of the agonist by removing the ligand by washing out.

Conclusions: The data in the present experiments demonstrate that prolonged administration of beta-3 adrenoceptor agonists produce time-dependent desensitization of the beta-3 adrenoceptor in the rat bladder. Recovery of receptor desensitization was achieved by removal or washing-out the agonist from the tissue, such that the receptor-mediated functional response in the bladder returns to baseline conditions.

Following either a one hr or three hr incubation with the EC₉₀ concentration of mirabegron, the ability of mirabegron to reduce the magnitude of EFS-mediated responses in rat bladder muscle was still attenuated markedly when the tissue was washed for only one or three hr. After 6 hr of washing post-incubation, the ability of mirabegron to reduce EFS was not significantly different than that seen in muscle strips that had only been exposed to an equivalent volume of vehicle and not to mirabegron. These data indicate that the effects of the exposure of the tissue to an EC₉₀ concentration of mirabegron produced desensitization of the responses mediated by the beta-3 adrenoceptor.

The EC₉₀ concentration of the beta-3 adrenoceptors agonists used in this study was selected because it reflects a clinically relevant concentration comparable to the Cₘₐₓ observed in patients. Beta-3 receptor desensitization appeared to occur rapidly, as only 1 hr incubation was necessary to produce marked inhibition of the beta-3 receptor mediated response.

The re-sensitization response occurred in a time-dependent manner, indicating the functional defect in the tissue was reversible, and recovery was completed within 6 hr. Such a time course of desensitization and re-sensitization is consistent with the time course that will be used for a pulsatile formulation administration of mirabegron in patients.

There was a clear difference between mirabegron and CL-316,243 at the low and middle concentrations of the concentration-response curve of the 1 and 3 hr washout curves. Following 1 or 3 hr of washout, the EFS-mediated response was actually potentiated to produce marked bladder contraction between the concentration range of 10-1000 nM. Interestingly, this is the concentration range that approximates the clinically relevant plasma levels of mirabegron in patients. The reason for the mirabegron-mediated potentiation on EFS-induced contraction of the rat bladder is currently unknown. Mirabegron may be producing effects through an alternative signaling pathways or other off-target effects may be potential mechanisms of this response.

CL-316,243 was used as a reference standard as a rodent selective beta-3 adrenoceptor agonist. Attenuation in the ability of CL-316,243 to reduce the magnitude of EFS responses in rat bladder muscle tissue was also seen after a three hr pre-incubation to the EC₉₀ concentration of CL-316,243. Following washout of CL-316,243 the recovery of the beta-3 adrenoceptor mediated response occurred in a time-dependent manner, as was seen with mirabegron.

In conclusion, the data in the present experiments demonstrate that prolonged administration of beta-3 adrenoceptor agonists can produce time-dependent desensitization of the beta-3 adrenoceptor-mediated responses in the rat bladder. Recovery of receptor desensitization and prevention of prolonged receptor desensitization can be achieved by removal of the agonist from the tissue, such that the receptor-mediated functional response returns to baseline conditions. Beta-3 receptor desensitization can be prevented by giving sufficient time between drug exposures for the tissue to recover. Therefore, prevention of prolonged administration of a beta-3 adrenoceptor agonists in patients with overactive bladder may be desirable in order to preserve and maximize therapeutic efficacy. Thus, the daily administration of a beta-3 adrenoceptor agonist that is formulated to occur in a pulsatile manner may be the viable approach for chronic treatment. Such an approach will minimize beta-3 adrenoceptor desensitization and promote recovery of desensitized receptors to become active.

### Example 7

### Multiparticulate Formulation for the Release of Mirabegron

A formulation utilizing mirabegron is proposed, wherein pellets containing mirabegron will form the basis for multiple releases of mirabegron to a patient in need. The formulation will contain at least two populations of pellets, wherein at least one population comprises an immediate release population and at least one population comprises a modified (i.e. sustained and/or delayed) release population. The immediate release pellets will release mirabegron immediately in the GI tract , whereas the modified release pellets will release mirabegron at a later time inside the GI tract. The modified release pellets may be coated with either a pH dependent (enteric) coating or a time dependent coating so as to delay the second release of mirabegron to the desired position in the GI tract. Both types of pellets may contain any physical form of mirabegron such as, for example, amorphous or crystalline solid. The pellets may be drug-layered pellets or matrix-type pellets.

### Example 8

### Drug Coated Spheres/Pellet with an Inert Core for the Release of Mirabegron

A formulation utilizing mirabegron is proposed, wherein spheres/pellets with an inert core containing mirabegron will form the basis for multiple releases of mirabegron to a patient in need. The formulation will contain at least two populations of spheres/pellets with an inert core, wherein at least one population comprises an immediate release population and at least one population comprises a modified (i.e. sustained and/or delayed) release population. The immediate spheres/pellets with an inert core will release mirabegron immediately in the GI tract, wherein the modified release spheres/pellets with an inert core will release mirabegron at a later time inside the GI tract. The modified release spheres/pellets with an inert core may be coated with either a pH dependent (enteric) coating or a time dependent coating so as to delay the second release of mirabegron to the desired position in the GI tract. Both types of spheres/pellets with an inert core may contain any physical form of mirabegron such as, for example, amorphous or crystalline solid.

### Example 9

### Bi-Layer Tablet for the Release of Mirabegron

A formulation utilizing mirabegron is proposed, wherein a bi-layer tablet containing mirabegron will form the basis for the multiple releases of mirabegron to a patient in need. The formulation will contain a tablet having both an immediate release layer and a modified release layer. The immediate layer will release mirabegron immediately in the GI tract, whereas the modified release layer will release mirabegron at a later time inside the GI tract. The modified release layer may be coated with either a pH dependent (enteric) coating or a time dependent coating so as to delay the second release of mirabegron to the desired position in the GI tract. Both types of layers may contain any physical form of mirabegron such as, for example, amorphous or crystalline solid.

### Example 10

### Matrix Tablet for the Release of Mirabegron

A formulation utilizing mirabegron is proposed, wherein a matrix tablet containing mirabegron will form the basis for the multiple releases of mirabegron to a patient in need thereof. The formulation will contain a well-mixed composite of drug(s) with rate-controlling excipients. Numerous sustained and/or delayed release tablets such as membrane controlled system, matrices with water soluble/insoluble polymers, and osmotic systems may be utilized. The tablet may contain either the amorphous form of mirabegron or the crystalline form. The delayed/sustained release can be achieved by applying a permeable or semipermeable membrane to the tablet core or by mixing the drug with excipient that is either a hydrophilic polymer with high viscosity and gel forming capability or a hydrophobic excipient that slows down the diffusion of drug molecule. An immediate release drug layer can be coated to the tablet that will be available for an early release in the GI tract, while the delayed release core will be designed to delay the drug release after a time period in a designated region of the GI tract.

### Example 11

### Multicore Tablet for the Release of Mirabegron

A formulation utilizing mirabegron is proposed, wherein a multicore tablet containing mirabegron will form the basis for the multiple releases of mirabegron to a patient in need thereof. The formulation will contain a multicore tablet that comprises multiple discrete cores consisting of at least one immediate release core and at least one delayed/sustained release core contained within the same tablet. The at least one immediate release core will be available for an early release in the GI tract, while the at least one delayed/sustained release core will be designed to delay the drug release after a time period in a designated region of the GI tract.

### Example 12

### Gastroretentive Delivery System for the Release of Mirabegron

A formulation utilizing mirabegron is proposed, wherein a gastroretentive oral dosage form containing mirabegron will form the basis for the multiple releases of mirabegron to a patient in need. The formulation will contain a tablet or capsule having both an immediate release and modified release component. The immediate layer will release mirabegron immediately in the GI tract , wherein the modified release layer will release mirabegron at a later time inside the GI tract. The gastroretentive oral dosage form may utilize mucoadhesive, swellable, high density or floating technologies to prolong residence time in the stomach thereby allowing a prolonged period for release of both first and second releases in the stomach or upper GI. Both releases may contain any physical form of mirabegron such as, for example, amorphous or crystalline solid.

Although the present disclosure has been described in considerable detail with reference to certain preferred versions thereof, other versions are possible. Therefore, the spirit and scope of the application should not be limited to the description of the preferred versions described herein.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the application, in addition to those described herein, will be apparent to those skilled in the art from the foregoing description. Such modifications are also intended to fall within the scope of the appended claims.

Throughout the above specification a number of references have been cited and or referred to it is to be understood that unless specifically noted, all references cited in the above specification and or referred to in the above specification are hereby incorporated by reference in their entirety.

What is claimed is:

1. A pharmaceutical composition comprising: a therapeutically effective amount of one or more beta-3 adrenoceptor agonists or a pharmaceutically acceptable salt thereof, wherein the pharmaceutical composition releases at least two pulses of one or more beta-3 adrenoceptor agonists, wherein a first pulse of one or more beta-3 adrenoceptor agonists achieves a first target Cₘₐₓ, a second pulse of one or more beta-3 adrenoceptor agonists achieves a second target Cₘₐₓ, a first target Cₘᵢₙ is achieved between the first pulse and the second pulse and a second Cₘᵢₙ is achieved after the second pulse, wherein the first target Cₘₐₓ and the second target Cₘₐₓ are different; with the proviso that the one or more beta-3 adrenoreceptor agonists is not solabegron.

2. The pharmaceutical composition according to claim 1, wherein the beta-3 adrenoceptor agonists is selected from the group consisting of: mirabegron; amibegron;
ritobegron; vibegron; L-742,791; L-796,568; TRK-380; LY-368,842; Ro40-2148;
pharmaceutically acceptable salts thereof; and combinations thereof

3. The pharmaceutical composition according to claim 1, further comprising one or more additional therapeutic agents useful for the treatment of LUTS, wherein the one more additional therapeutic agents are selected from the groups consisting of: antimuscarinic agents; alpha adrenoceptor blockers; 5-alpha reductases; and phosphodiesterase-5 inhibitors.

4. The pharmaceutical composition according to claim 3, wherein the one or more additional therapeutic agents may be administered prior to, simultaneously with, or following the administration of the pharmaceutical composition comprising one or more beta-3 adrenoceptor agonists.

5. The pharmaceutical composition according to claim 1, wherein the pharmaceutical composition achieves a concentration of the one or more beta-3 adrenoceptor agonists that is below 1 μg/ml for about 6-9 hours over a twenty-four hour period.

6. A pharmaceutical composition for the delivery of one or more beta-3 adrenoceptor agonists, comprising:
a. at least one immediate release composition, comprising one or more beta-3 adrenoceptor agonists or a pharmaceutically acceptable salt thereof, and at least one pharmaceutically acceptable carrier or diluent; and
b. at least one modified release composition, comprising one or more beta-3 adrenoceptor agonists or a pharmaceutically acceptable salt thereof, and at least one pharmaceutically acceptable carrier or diluent; with the proviso that the one or more beta-3 adrenoreceptor agonists is not solabegron.

7. The pharmaceutical composition according to claim 6, wherein the beta-3 adrenoceptor agonist is selected from the group consisting of: mirabegron; amibegron;
ritobegron; vibegron; L-742,791; L-796,568; TRK-380; LY-368,842; Ro40-2148; or a pharmaceutically acceptable salt thereof; and combinations thereof.

8. The pharmaceutical composition according to claim 6, further comprising one or more additional therapeutic agents useful for the treatment of LUTS, wherein the one more additional therapeutic agents are selected from the groups consisting of: antimuscarinic agents; alpha adrenoceptor blockers; 5-alpha reductases; and phosphodiesterase-5 inhibitors.

9. The pharmaceutical composition according to claim 8, wherein the one or more additional therapeutic agents may be administered prior to, simultaneously with, or following the administration of the pharmaceutical composition comprising the one or more beta-3 adrenoceptor agonists.

10. The pharmaceutical composition according to claim 6, wherein the pharmaceutical composition achieves a concentration of the one or more beta-3 adrenoceptor agonists that is below 1 μg/ml for about 6-9 hours over a twenty-four hour period.

11. A once-daily treatment for LUTS that achieves a desired blood serum Cₘₐₓ while also not desensitizing the beta-3 adrenoceptor, comprising: a pharmaceutical composition, comprising a therapeutically effective amount of one or more beta-3 adrenoceptor agonists or a pharmaceutically acceptable salt thereof, wherein the pharmaceutical composition releases at least two pulses of one or more beta-3 adrenoceptor agonists, wherein a first pulse of one or more beta-3 adrenoceptor agonists achieves a first target Cₘₐₓ, a second pulse of one or more beta-3 adrenoceptor agonists achieves a second target Cₘₐₓ, a first target Cₘᵢₙ is achieved between the first pulse and the second pulse and a second Cₘᵢₙ is achieved after the second pulse, wherein the first target Cₘₐₓ and the second target Cₘₐₓ are different; with the proviso that the one or more beta-3 adrenoreceptor agonists is not solabegron.

12. The treatment according to claim 11, wherein the beta-3 adrenoceptor agonists is selected from the group consisting of: mirabegron; amibegron; ritobegron; vibegron; L-742,791; L-796,568; TRK-380; LY-368,842; Ro40-2148; and combinations thereof or a pharmaceutically acceptable salt thereof.

13. The treatment according to claim 11, further comprising administering one or more additional therapeutic agents useful for the treatment of LUTS, wherein the one more additional therapeutic agents are selected from the groups consisting of: antimuscarinic agents; alpha adrenoceptor blockers; 5-alpha reductases; and phosphodiesterase-5 inhibitors.

14. The treatment according to claim 13, wherein the one or more additional therapeutic agents may be administered prior to, simultaneously with, or following the administration of the pharmaceutical composition comprising one or more beta-3 adrenoceptor agonists.

15. A once-daily treatment for LUTS that achieves a desired blood serum Cₘₐₓ while also not desensitizing the beta-3 adrenoceptor, comprising:
a. at least one immediate release composition, comprising one or more beta-3 adrenoceptor agonists and at least one pharmaceutically acceptable carrier or diluent; and
b. at least one modified release composition, comprising one or more beta-3 adrenoceptor agonists and at least one pharmaceutically acceptable carrier or diluent; with the proviso that the one or more beta-3 adrenoreceptor agonists is not solabegron..

16. The treatment according to claim 15, wherein the beta-3 adrenoceptor agonists is selected from the group consisting of: mirabegron; amibegron; ritobegron; vibegron; L-742,791; L-796,568; TRK-380; LY-368,842; Ro40-2148; and combinations thereof.

17. The treatment according to claim 15, further comprising administering one or more additional therapeutic agents useful for the treatment of LUTS, wherein the one more additional therapeutic agents are selected from the groups consisting of: antimuscarinic agents; alpha adrenoceptor blockers; 5-alpha reductases; and phosphodiesterase-5 inhibitors.

18. The treatment according to claim 17, wherein the one or more additional therapeutic agents may be administered prior to, simultaneously with, or following the administration of the pharmaceutical composition comprising one or more beta-3 adrenoceptor agonists.

19. A method of treating LUTS in a patient in need thereof, comprising
a.) administering a pharmaceutical composition comprising, a therapeutically effective amount of a beta-3 adrenoceptor agonist or a pharmaceutically acceptable salt thereof and at least one pharmaceutically acceptable carrier or diluent on days 1, 3, 5, 7, 9, 11, 13, 15, 17, 19. 21, 23, 25 and 27 of a 28 day dosing cycle; and
b.) administering a pharmaceutical composition comprising, a therapeutically effective amount of anti-muscarinic agent or a pharmaceutically acceptable salt thereof and at least one pharmaceutically acceptable carrier or diluent on days 2, 4, 6, 8, 10, 12, 14, 16, 18, 20, 22, 24, 26 and 28 of a 28 day dosing cycle
to a patient in need thereof.

20. The method of claim 19, wherein the beta-3 adrenoceptor agonist is selected from the group consisting of: amibegron; mirabegron; ritobegron; vibegron; solabegron, L-742,791; L-796,568; TRK-380; LY-368,842; Ro40-2148 and pharmaceutically acceptable salts thereof.

21. The method of claim 19, wherein the anti-muscarinic agent is selected from the group consisting of: tolterodine; oxybutynin; trospium; solifenacin; darifenacin; propiverine; fesoterodine; and pharmaceutically acceptable salts thereof

22. The method of claim 19, wherein the pharmaceutical composition comprising, a therapeutically effective amount of a beta-3 adrenoceptor agonist and at least one pharmaceutically acceptable carrier or diluent is an immediate-release composition.

23. The method of claim 19, wherein the pharmaceutical composition comprising, a therapeutically effective amount of a beta-3 adrenoceptor agonist and at least one pharmaceutically acceptable carrier or diluent is a modified-release composition.

24. The method of claim 19, wherein the pharmaceutical composition comprising, a therapeutically effective amount of a beta-3 adrenoceptor agonist and at least one pharmaceutically acceptable carrier or diluent comprises at least two pulses of the beta-3 adrenoceptor, wherein a first pulse of beta-3 adrenoceptor achieves a first target Cₘₐₓ, a second pulse of beta-3 adrenoceptor achieves a second target Cₘₐₓ, a first target Cₘᵢₙ is achieved between the first pulse and the second pulse, and a second Cₘᵢₙ is achieved after the second pulse.

25. The method of claim 19, wherein the pharmaceutical composition comprising, a therapeutically effective amount of an anti-muscarinic agent and at least one pharmaceutically acceptable carrier or diluent is an immediate-release composition.

26. The method of claim 19, wherein the pharmaceutical composition comprising, a therapeutically effective amount of an anti-muscarinic agent and at least one pharmaceutically acceptable carrier or diluent is a modified-release composition.

27. A method of treating LUTS, in a patient in need thereof, comprising administering to the patient in need thereof, a beta-3 adrenoceptor agonist or pharmaceutically acceptable salt thereof and an antimuscarinic agent or pharmaceutically acceptable salt thereof on alternating days.

28. A consumer pack for treating LUTS, comprising
a.) a pharmaceutical composition comprising, a therapeutically effective amount of a beta-3 adrenoceptor agonist or a pharmaceutically acceptable salt thereof and at least one pharmaceutically acceptable carrier or diluent on days 1, 3, 5, 7, 9, 11, 13, 15, 17, 19, 21, 23, 25 and 27 of a 28 day dosing cycle;
b.) a pharmaceutical composition comprising, a therapeutically effective amount of anti-muscarinic agent or a pharmaceutically acceptable salt thereof and at least one pharmaceutically acceptable carrier or diluent on days 2, 4, 6, 8, 10, 12, 14, 16, 18, 20, 22, 24, 26 and 28 of a 28 day dosing cycle; and
c.) a packet, comprising 28 separate containers arranged in a 28 day pattern, wherein the pharmaceutical composition comprising the beta-3 adrenoceptor and the pharmaceutical composition comprising the anti-muscarinic agent are in their appropriate containers.

29. The consumer pack of claims 28, wherein the beta-3 adrenoceptor agonist is selected from the group consisting of: amibegron; mirabegron; ritobegron; vibegron; solabegron, L-742,791; L-796,568; TRK-380; LY-368,842; Ro40-2148 and pharmaceutically acceptable salts thereof

30. The consumer pack of claims 28, wherein the anti-muscarinic agent is selected from the group consisting of: tolterodine; oxybutynin; trospium; solifenacin; darifenacin; propiverine; fesoterodine; and pharmaceutically acceptable salts thereof.
